(19) United States
(12) Patent Application Publication
(10) Pub. No.: US 2023/0013253 A1
(43) Pub. Date: Jan. 19, 2023

(54) COMPOSTIONS AND METHODS FOR NUCLEIC ACID TRANSFECTION USING CATIONIC POLYMERS AND STABILIZERS

(71) Applicant: Pfizer Inc., New York, NY (US)

(72) Inventors: Saurabh Kant Bhardwaj, Chesterfield, MO (US); Chia Hung Chu, Chesterfield, MO (US)

(21) Appl. No.: 17/756,951

(22) PCT Filed: Dec. 16, 2020
(86) PCT No.: PCT/IB2020/062030

§ 371 Date: Jun. 6, 2022

Related U.S. Application Data
(60) Provisional application No. 62/950,464, filed on Dec. 19, 2019.

(51) Int. Cl. C12N 15/86

(52) U.S. Cl. CPC C12N 15/86; C12N 2750/14052; C12N 2750/14043

(57) ABSTRACT

Provided are compositions and methods for stabilizing a transfection cocktail containing DNA-cationic polymer complexes for an extended time, while maintaining high transfection efficiency. Such stabilized transfection cocktail can be used to generate transfected cells that can produce, for example, rAAV vectors on a large scale without impacting the key attributes of the virus production, such as, titer, DNA packaged rAAV particle fraction, and rAAV vector purification profile.

### CROSS-REFERENCE TO RELATED APPLICATIONS

This application claims the benefit of U.S. Provisional Application No. 62/950,464 filed Dec. 19, 2019, which is hereby incorporated by reference in its entirety.

### FIELD OF THE INVENTION

The present invention relates to the field of cell transfection with nucleic acids (e.g., plasmids). Specifically, the present invention relates to compositions and methods for producing transfected cells, said cells optionally producing recombinant adeno-associated viral (rAAV) vectors that are useful for gene therapy.

### BACKGROUND

Transient transfection of cells for producing various biotherapeutic molecules, including rAAV vectors, has picked up pace in the recent past. This approach allows production of material on a large-scale for both preliminary studies and clinical trials without the need for creating stable cell lines which typically involves a longer and more arduous process. Various transfection reagents, such as cationic polymers, have been identified with the ability to transfect cells. Cationic polymers, such as polyethylenimine (PEI) form complexes with plasmid DNA (referred to as “polyplexes”) and can transfect cells most efficiently with low toxicity to cells.

However, a major disadvantage of polyplexes is their lack of colloidal stability which over time leads to aggregation of the DNA-PEI complexes into large particles, particularly at high DNA concentrations, thereby severely compromising their ability to transfect cells (See, for example, Sharma et al., (2005) Mechanistic studies on aggregation of polyethylenimine-DNA complexes and its prevention, Biotechnol Bioeng. 90(5):614-20; Ito et al. (2008). Efficient in vivo gene transfection by stable DNA/PEI complexes coated by hyaluronic acid, journal of Drug Targeting, 16:4, 276-281). A number of approaches have been tried to overcome this problem, including conjugating the cationic polymer to a poly(ethylene glycol) (PEG) moiety (See, for example, Wolfert et al., (1996) Characterization of vectors for gene therapy formed by self- assembly of DNA with synthetic block co-polymers, Hum Gene Ther., 7(17):2123-33). Modification of the cationic polymer with PEG, however, has a negative impact on transfection efficiency.

It is, therefore, apparent that there is a need for compositions and methods that provide stable DNA-PEI complexes for an extended time, while maintaining high transfection efficiency. This would allow for the large-scale manufacture of cells that can produce large quantities of biotherapeutic molecules, such as high titer rAAV vectors.

All publications, patents, and patent applications cited herein are hereby incorporated by reference herein in their entirety for all purposes to the same extent as if each individual publication, patent, and patent application were specifically and individually indicated to be so incorporated by reference. If one or more of the incorporated literatures and similar materials differs from or contradicts this application, including but not limited to defined terms, term usage, described techniques, or the like, this application controls.

### SUMMARY OF THE INVENTION

Disclosed herein are compositions and methods for stabilizing a transfection cocktail containing DNA-cationic polymer complexes for an extended time, while maintaining high transfection efficiency. Such stabilized transfection cocktail can be used to generate transfected cells that can produce, for example, rAAV vectors on a large scale without impacting the key attributes of the virus production, such as, titer, DNA packaged rAAV particle fraction, and rAAV vector purification profile.

Disclosed and exemplified herein are compositions and methods for producing transfected cells. Those skilled in the art will recognize or be able to ascertain using no more than routine experimentation, many equivalents to the specific embodiments of the invention described herein. Such equivalents are intended to be encompassed by the following embodiments (E).

E1. A method for transfecting cells with one or more nucleic acids, comprising the following steps:
(i) preparing a transfection cocktail comprising one or more cationic polymers, a stabilizer and one or more nucleic acids,
(ii) contacting the transfection cocktail prepared in step (i) with cells to be transfected to form a mixture, and
(iii) incubating the mixture of step (ii) thereby transfecting the cells with the one or more nucleic acids.
E2. A method for making cells that produce recombinant adeno-associated viral (rAAV) vector, comprising the following steps:
(i) preparing a transfection cocktail comprising one or more cationic polymers, a stabilizer and one or more nucleic acids,
(ii) contacting the transfection cocktail prepared in step (i) with cells to be transfected to form a mixture, and
(iii) incubating the mixture of step (ii) thereby making transfected cells that produce rAAV vector.
E3. A method for increasing transfection of cells with one or more nucleic acids, comprising the following steps:
(i) preparing a transfection cocktail comprising one or more cationic polymers, a stabilizer and one or more nucleic acids,
(ii) contacting the transfection cocktail prepared in step (i) with cells to be transfected to form a mixture, and
(iii) incubating the mixture of step (ii), whereby transfection of the cells with the one or more nucleic acids is increased as compared to the transfection of the cells performed under the same conditions but in the absence of the stabilizer.
E4. A method for producing high titer recombinant adeno-associated viral (rAAV) vector, comprising the following steps:
(i) preparing a transfection cocktail comprising one or more cationic polymers, a stabilizer and one or more nucleic acids,
(ii) contacting the transfection cocktail prepared in step (i) with cells to be transfected to form a mixture,
(iii) incubating the mixture of step (ii) to make transfected cells that produce rAAV vector, and
(iv) isolating and/or purifying rAAV vector from the transfected cells produced in step (iii), wherein the time between initiation of step (i) and completion of step (ii) is greater than 10 seconds, and wherein the rAAV titer is increased by at least 2-fold and/or by at least 5% as compared to the rAAV titer produced under the same conditions but in the absence of the stabilizer.
E5. The method as set forth in any one of E1-E4, wherein the time between initiation of step (i) and completion of step (ii) is between about 10 seconds to about 10 days.
E6. The method as set forth in E5, wherein the time between initiation of step (i) and completion of step (ii) is between about 15 seconds to about 5 days, about 30 seconds to about 2 days, about 60 seconds to about 1 day, about 90 seconds to about 10 hours, about 90 seconds to about 8 hours, about 2 minutes to about 4 hours, about 4 minutes to about 2 hours, about 6 minutes to about 1 hour, or about 10 minutes to about 30 minutes.
E7. The method as set forth in any one of E1-E6, wherein the time between initiation of step (i) and completion of step (ii) is more than about 1 minute, more than about 2 minutes, more than about 6 minutes, more than about 8 minutes, more than about 10 minutes, more than 15 minutes, more than about 30 minutes, more than about 1 hour, more than about 2 hours, more than about 6 hours, more than about 8 hours, more than about 10 hours, more than about 12 hours, more than about 24 hours, or more than 2 days.
E8. The method as set forth in any one of E1-E7, wherein the time between initiation of step (i) and completion of step (ii) is more than 10 minutes.
E9. The method as set forth in any one of E1-E8, wherein the time between initiation of step (i) and completion of step (ii) is about 30 minutes.
E10. The method as set forth in any one of E1-E9, wherein the transfection cocktail of step (i) is incubated from about 10 seconds to about 10 days, about 15 seconds to about 5 days, about 30 seconds to about 2 days, about 60 seconds to about 1 day, about 90 seconds to about 10 hours, about 90 seconds to about 8 hours, about 2 minutes to about 4 hours, about 4 minutes to about 2 hours, about 6 minutes to about 1 hour, or about 10 minutes to about 30 minutes prior to step (ii).
E11. The method as set forth in any one of E1-E10, wherein the transfection cocktail of step (i) is incubated for more than about 1 minute, more than about 2 minutes, more than about 6 minutes, more than about 8 minutes, more than about 10 minutes, more than 15 minutes, more than about 30 minutes, more than about 1 hour, more than about 2 hours, more than about 6 hours, more than about 8 hours, more than about 10 hours, more than about 12 hours, more than about 24 hours, or more than 2 days prior to step (ii).
E12. The method as set forth in any one of E1-E11, wherein the transfection cocktail is prepared by first mixing the one or more cationic polymers with the stabilizer to form a resultant mixture which is then added to the one or more nucleic acids.
E13. The method as set forth in any one of E1-E11, wherein the transfection cocktail is prepared by first mixing the stabilizer with the one or more nucleic acids to form a resultant mixture which is then added to the one or more cationic polymers.
E14. The method as set forth in E13, wherein the cells are at a high cell density (e.g., more than about 18×10⁶ cells/mL) when contacted with the transfection cocktail in step (ii).
E15. The method as set forth in any one of E1-E11, wherein the transfection cocktail is prepared by first mixing the one or more cationic polymers with the one or more nucleic acids to form a resultant mixture which is then added to the stabilizer.
E16. The method as set forth in any one of E1-E15, wherein the cells are at a cell density of at least 1×10⁵ cells/mL, at least 2×10⁵ cells/mL, at least 4×10⁵ cells/mL, at least 6×10⁵ cells/mL, at least 8×10⁵ cells/mL, at least 0.5×10⁶ cells/mL, at least 1×10⁶ cells/mL, at least 2×10⁶ cells/mL, at least 4×10⁶ cells/mL, at least 6×10⁶ cells/mL, at least 8×10⁶ cells/mL, at least 10×10⁶ cells/mL, at least 12×10⁶ cells/mL, at least 14×10⁶ cells/mL, at least 16×10⁶ cells/mL, at least 18×10⁶ cells/mL, at least 20×10⁶ cells/mL, at least 22×10⁶ cells/mL, at least 24×10⁶ cells/mL, at least 26×10⁶ cells/mL, at least 28×10⁶ cells/mL, at least 30×10⁶ cells/mL, at least 32×10⁶ cells/mL, at least 34×10⁶ cells/mL, at least 36×10⁶ cells/mL, at least 38×10⁶ cells/mL, at least 40×10⁶ cells/mL, at least 42×10⁶ cells/mL, at least 44×10⁶ cells/mL, at least 46×10⁶ cells/mL, at least 48×10⁶ cells/mL, at least 50×10⁶ cells/mL, or at least 52×10⁶ cells/mL when contacted with the transfection cocktail in step (ii).
E17. The method as set forth in E16, wherein the cells are at a density of at least 12×10⁶ cells/mL, at least 14×10⁶ cells/mL, at least 16×10⁶ cells/mL, at least 18×10⁶ cells/mL, at least 20×10⁶ cells/mL, at least 22×10⁶ cells/mL, at least 24×10⁶ cells/mL, at least 26×10⁶ cells/mL, at least 28×10⁶ cells/mL, or at least 30×10⁶ cells/mL, at least 32×10⁶ cells/mL, at least 34×10⁶ cells/mL, at least 36×10⁶ cells/mL, at least 38×10⁶ cells/mL, at least 40×10⁶ cells/mL, at least 42×10⁶ cells/mL, at least 44×10⁶ cells/mL, at least 46×10⁶ cells/mL, or at least 48×10⁶ cells/mL, when contacted with the transfection cocktail in step (ii).
E18. The method as set forth in E13, wherein the cells are at a density of at least 24×10⁶ cells/mL, at least 26×10⁶ cells/mL, at least 28×10⁶ cells/mL, at least 30×10⁶ cells/mL, at least 32×10⁶ cells/mL, at least 34×10⁶ cells/mL, at least 36×10⁶ cells/mL, at least 38×10⁶ cells/mL, at least 40×10⁶ cells/mL, at least 42×10⁶ cells/mL, at least 44×10⁶ cells/mL, at least 46×10⁶ cells/mL, or at least 48×10⁶ cells/mL when contacted with the transfection cocktail in step (ii).
E19. The method as set forth in E18, wherein the cells are at a density of at least 24×10⁶ cells/mL when contacted with the transfection cocktail in step (ii).
E20. The method as set forth in any one of E1-E19, wherein the one or more cationic polymers are used in an amount of about 0.05 μg per million cells, about 0.1 μg per million cells, about 0.2 μg per million cells, about 0.4 μg per million cells, about 0.6 μg per million cells, about 0.8 μg per million cells, about 1.0 μg per million cells, about 1.5 μg per million cells, about 2 μg per million cells, about 2.1 μg per million cells, about 2.2 μg per million cells, about 2.3 μg per million cells, about 2.4 μg per million cells, about 2.5 μg per million cells, about 2.6 μg per million cells, about 2.7 μg per million cells, about 2.8 μg per million cells, about 2.9 μg per million cells, about 3.0 μg per million cells, about 3.5 μg per million cells, about 4.0 μg per million cells, about 4.5 μg per million cells, about 5 μg per million cells, about 5.5 μg per million cells, about 6.0 μg per million cells, about 6.5 μg per million cells, about 7.0 μg per million cells, about 7.5 μg per million cells, about 8.0 μg per million cells, about 10 μg per million cells.
E21. The method as set forth in E20, wherein the one or more cationic polymers are used in an amount of about 2.2 μg per million cells.
E22. The method as set forth in any one of E1-E21 wherein the amount of stabilizer in the transfection cocktail is about 1%, about 2%, about 2.5%, about 5%, about 7.5%, about 10%, about 12.5%, about 15%, about 17.5%, about 20%, about 25%, about 30%, about 40%, about 50%, about 60%, about 70%, about 75%, about 80%, about 90%, about 100%, about 200%, about 300%, about 400%, or about 500% relative to the amount of the one or more cationic polymers.
E23. The method as set forth in any one of E1-E22, wherein the amount of stabilizer in the transfection cocktail is about 0.01 fold, about 0.05 fold, about 0.1 fold, about 0.2-fold, about 0.4 fold, about 0.6 fold, about 0.8 fold, about 1-fold, about 5-fold, about 10-fold, about 100 fold, about 200 fold, about 300 fold, about 400 fold, about 500 fold, or about 600 fold relative to the amount of the one or more cationic polymers.
E24. The method as set forth in E22, wherein the amount of stabilizer in the transfection cocktail is about 7.5%, about 10%, about 12.5%, about 15%, about 17.5%, about 20%, about 25%, about 30% or about 40% relative to the amount of the one or more cationic polymers.
E25. The method as set forth in any one of E1-E24, wherein the amount of stabilizer in the transfection cocktail is about 0.01 μg, about 0.02 μg, about 0.04 μg, about 0.06 μg, about 0.08 μg, about 1.0 μg, about 1.2 μg, about 1.4 μg, about 1.6 μg, about 1.8 μg, about 2.0 μg, about 2.2 μg, about 2.4 μg, about 2.6 μg, about 2.8 μg, about 3.0 μg, about 3.2 μg, about 3.4 μg, about 3.6 μg, about 3.8 μg, about 4.0 μg, about 4.2 μg, about 4.6 μg, about 4.8 μg, about 5.0 μg, about 5.5 μg, about 6.0 μg, about 6.5 μg, about 7 μg, about 7.5 μg, about 8.0 μg, about 8.5 μg, about 9.0 μg, about 9.5 μg, about 10 μg, about 12 μg, about 14 μg, about 16 μg, about 18 μg, about 20 μg, about 40 μg, about 60 μg, about 80 μg, about 100 μg, about 0.1 mg, about 1 mg, about 2 mg, about 4 mg, about 6 mg, about 8 mg or about 10 mg.
E26. The method as set forth in any one of E1-E25, wherein the cells are at a low cell density (e.g., less than about 18×10⁶ cells/mL) when contacted with the transfection cocktail in step (ii) and the amount of stabilizer in the transfection cocktail is about 7.5% to about 10% relative to the amount of the one or more cationic polymers.
E27. The method as set forth in any one of E1-E25, wherein the cells are at a high cell density (e.g., more than about 18×10⁶ cells/mL) when contacted with the transfection cocktail in step (ii) and the amount of stabilizer in the transfection cocktail is about 15% to about 30% relative to the amount of the one or more cationic polymers.
E28. The method as set forth in E26, wherein the cells at a density of about 12×10⁶ cells/mL when contacted with the transfection cocktail in step (ii) and the amount of stabilizer in the transfection cocktail is about 10% relative to the amount of the one or more cationic polymers.
E29. The method as set forth in E27, wherein the cells are at a density of about 24×10⁶ cells/mL when contacted with the transfection cocktail in step (ii) and the amount of stabilizer in the transfection cocktail is about 25% relative to the amount of the one or more cationic polymers.
E30. The method as set forth in any one of E1-E29, wherein the amount of stabilizer in the transfection cocktail is insufficient to transfect the cells when used alone (i.e., without one or more cationic polymers).
E31. The method as set forth in any one of E1-E30, wherein the one or more nucleic acids are used in an amount of about 0.05 μg per million cells, about 0.1 μg per million cells, about 0.2 μg per million cells, about 0.4 μg per million cells, about 0.5 μg per million cells, about 0.6 μg per million cells, about 0.8 μg per million cells, about 1.0 μg per million cells, about 1.5 μg per million cells, about 2.0 μg per million cells, about 2.5 μg per million cells, about 3.0 μg per million cells, about 3.5 μg per million cells, about 4.0 μg per million cells, about 4.5 μg per million cells, about 5 μg per million cells, about 5.5 μg per million cells, about 6.0 μg per million cells, about 6.5 μg per million cells, about 7.0 μg per million cells, about 7.5 μg per million cells, about 8.0 μg per million cells, about 8.5 μg per million cells, about 9.0 μg per million cells, about 9.5 μg per million cells, or about 10 μg per million cells.
E32. The method as set forth in E31, wherein the one or more nucleic acids are used in an amount of about 1.0 μg per million cells.
E33. The method as set forth in any one of E1-E32, wherein the one or more nucleic acids and the one or more cationic polymers are used in a weight (or molar) ratio in the range of about 1:0.01 to about 1:100, or in a weight (or molar) ratio of about 0.01:1 to about 100:1.
E34. The method as set forth in any one of E1-E33, wherein the transfection cocktail in step (i) is prepared at from about 4° C. to about room temperature.
E35. The method as set forth in E34, wherein the transfection cocktail in step (i) is prepared at about room temperature.
E36. The method as set forth in any one of E10-E35, wherein the transfection cocktail is incubated at about 4° C. to about room temperature prior to step (ii).
E37. The method as set forth in E36, wherein the transfection cocktail is incubated at about room temperature prior to step (ii).
E38. The method as set forth in any one of E12-E37, wherein the resultant mixture is added to the one or more nucleic acids, the one or more cationic polymers or the stabilizer with no mixing.
E39. The method as set forth in any one of E12-E37, wherein the resultant mixture is added to the one or more nucleic acids, the one or more cationic polymers or the stabilizer and mixed at about 10 revolutions per minute (rpm), about 20 rpm, about 25 rpm, about 30 rpm, about 35 rpm, about 40 rpm, about 45 rpm, about 50 rpm, about 55 rpm, about 60 rpm, about 65 rpm, about 70 rpm, about 75 rpm, about 80 rpm, about 85 rpm, about 90 rpm, about 95 rpm, about 100 rpm, about 110 rpm, about 120 rpm, about 130 rpm, about 140 rpm, about 150 rpm, about 200 rpm, about 300 rpm, about 400 rpm or about 500 rpm.
E40. The method as set forth in E39, wherein the resultant mixture is is added to the one or more nucleic acids, the one or more cationic polymers or the stabilizer and mixed at about 120 rpm.
E41. The method as set forth in any one of E1-E40, wherein the mixture in step (iii) is incubated for about 15 minutes to about 150 hours to make transfected cells.
E42. The method as set forth in E41, wherein the mixture in step (iii) is incubated for about 15 minutes, 30 minutes, 1 hour, 2 hours, 3 hours, 4 hours, 5 hours, 6 hours, 7 hours, 8 hours, 9 hours, 10 hours, 20 hours, 40 hours, 60 hours, 80 hours, 100 hours, 120 hours, 140 hours or 150 hours to make transfected cells.
E43. The method as set forth in E42, wherein the mixture in step (iii) is incubated for 1 hour, 2 hours, 3 hours, 4 hours, or 5 hours to make transfected cells.
E44. The method as set forth in any one of E1-E43, wherein the one or more cationic polymers is selected from the group consisting of chitosan, poly-L-lysine, polyamine (PA), polyalkylenimine (PAI), polyethylenimine (PEI), poly[a-(-aminobutyl)-L-glycolic acid], polyamidoamine, poly(2-dimethylamino)ethyl methacrylate, polyhistidine, polyarginine, poly(4-vinylpyridine), poly(vinylamine) and poly(4-vinyl-N-alkyl pyridinium halide).
E45. The method as set forth in any one of E1-E44, wherein the cationic polymer is branched or linear.
E46. The method as set forth in any one of E1-E45, wherein the cationic polymer has a molecular weight ranging from about 500 Da to about 160,000 Da and/or about 2,500 Da to about 250,000 Da in free base form.
E47. The method as set forth in any one of E1-E46, wherein the one or more cationic polymers is polyethylenimine (PEI).
E48. The method as set forth in E47, wherein the PEI comprises branched PEI with a molecular weight from about 2,000 Da to about 60,000 Da.
E49. The method as set forth in E47, wherein the PEI comprises linear PEI with a molecular weight from about 2,000 Da to about 60,000 Da
E50. The method as set forth in E49, wherein the cationic polymer comprises hydrolyzed linear PEI with a molecular weight of about 40,000 Da and/or about 22,000 Da molecular weight in free base form.
E51. The method as set forth in any one of E47-E50, wherein the PEI has a fully depropionylated structure.
E52. The method as set forth in any one of E47-E52, wherein the PEI is PEImax.
E53. The method as set forth in any one of E1-E52, wherein the stabilizer comprises a cationic polymer grafted with a neutral moiety.
E54. The method as set forth in E53, wherein the neutral moiety comprises poly(ethylene glycol) (PEG) or albumin.
E55. The method as set forth in any one of E1-E54, wherein the stabilizer comprises a cationic polymer grafted with a poly(ethylene glycol) (PEG) moiety.
E56. The method as set forth in any one of E54-E55, wherein PEG has a molecular weight from about 250 Da to 35,000 Da.
E57. The method as set forth in any one of E53-E56, wherein the cationic polymer grafted with the neutral moiety (e.g., PEG) is selected from the group consisting of chitosan, poly-L-lysine (pLL), polyamine (PA), polyalkylenimine (PAI), polyethylenimine (PEI), poly[a-(-aminobutyl)-L-glycolic acid], polyamidoamine, poly(2-dimethylamino)ethyl methacrylate, polyhistidine, polyarginine, poly(4-vinylpyridine), poly(vinylamine) and poly(4-vinyl-N-alkyl pyridinium halide).
E58. The method as set forth in E57, wherein the cationic polymer has a molecular weight ranging from about 500 Da to about 160,000 Da and/or about 2,500 Da to about 250,000 Da in free base form
E59. The method as set forth in any one of E53-E58, wherein the cationic polymer is branched or linear.
E60. The method as set forth in any one of E53-E59, wherein the cationic polymer is PEI.
E61. The method as set forth in any one of E53-E60, wherein the stabilizer comprises branched PEI with a molecular weight from about 2,000 Da to about 60,000 Da grafted with PEG having a molecular weight from about 250 Da to about 35,000 Da.
E62. The method as set forth in E61, wherein the stabilizer comprises branched PEI with a molecular weight of about 20,000 Da to about 25,000 Da grafted with PEG having a molecular weight from about 250 Da to about 5,000 Da.
E63. The method as set forth in E62, wherein the stabilizer comprises branched PEI with a molecular weight of about 25,000 Da grafted with PEG having a molecular weight of about 5,000 Da.
E64. The method as set forth in E62, wherein the stabilizer comprises branched PEI with a molecular weight of about 25,000 Da grafted with PEG having a molecular weight of about 500 Da.
E65. The method as set forth in any one of E53-E60, wherein the stabilizer comprises linear PEI with a molecular weight from about 2,000 Da to about 60,000 Da grafted with PEG having a molecular weight from about 250 Da to 35,000 Da.
E66. The method as set forth in E65, wherein the stabilizer comprises linear PEI with a molecular weight of about 20,000 Da grafted with PEG having a molecular weight of about 5,000 Da.
E67. The method as set forth in E65, wherein the stabilizer comprises linear PEI with a molecular weight of about 20,000 Da grafted with PEG having a molecular weight of about 500 Da.
E68. The method as set forth in any one of E53-E59, wherein the stabilizer comprises pLL with a molecular weight of about 26000 Da conjugated to PEG having a molecular weight of about 5000 Da.
E69. The method as set forth in any one of E54-E68, wherein the number of PEG moieties grafted per molecule of cationic polymer is 1, 5, 10, 15, 20, 25, 30, 35, 40, 45, 50, 55, 60, 65, 70, 75, 80, 85, 90, 95, 100 or more.
E70. The method as set forth in E69, wherein the number of PEG moieties grafted per molecule of cationic polymer is 1, 5, 15 or 55.
E71. The method as set forth in any one of E1-E70, wherein the one or more nucleic acid molecules comprise one or more plasmids.
E72. The method as set forth in E71, wherein the one or more nucleic acid molecules comprise (a) one or more plasmids comprising nucleic acids encoding AAV packaging proteins and/or nucleic acids encoding helper proteins, and/or (b) a plasmid comprising a nucleic acid encoding a transgene of interest.
E73. The method as set forth in E72, wherein the one or more plasmids of (a) comprise a first plasmid comprising the nucleic acids encoding AAV packaging proteins and a second plasmid comprising the nucleic acids encoding helper proteins.
E74. The method as set forth in E73, wherein the molar ratio of the plasmid comprising the transgene to the first plasmid comprising the nucleic acids encoding AAV packaging proteins to the second plasmid comprising the nucleic acids encoding helper proteins is in the range of about 1-10:1:1, or 1:1-10:1, or 1:1:1-10.
E75. The method as set forth in any one of E72-E74, wherein the encoded AAV packaging proteins comprise AAV rep and/or AAV cap proteins.
E76. The method as set forth in any one of E72-E75, wherein the encoded helper proteins comprise adenovirus E2 and/or E4, VARNA proteins, and/or non-AAV helper proteins.
E77. The method as set forth in any one of E72-E76, wherein the transgene encodes a wild type or functional variant blood clotting factor, mini-dystrophin, C1 esterase inhibitor, copper transporting P-type ATPase (ATP7B), or copper-zinc superoxide dismutase 1 (SOD1).
E78. The method as set forth in E77, wherein the wild type or functional variant blood clotting factor is Factor VII, Factor VIII or Factor IX.
E79. The method as set forth in any one of E1-E3, E5-E78, further comprising step (iv) culturing, expanding, isolating or selecting for cells that have been transfected with the one or more nucleic acids.
E80. The method as set forth in any one of E1-E3, E5-E78, further comprising step (iv) harvesting the transfected cells produced in step (iii) and/or culture medium from the transfected cells produced in step (iii) to produce a cell and/or culture medium harvest.
E81. The method as set forth in any one of E1-E3, E5-E78, further comprising step (iv) isolating and/or purifying rAAV vector from the transfected cells produced in step (iii).
E82. The method as set forth in any one of E1-E81, wherein transfection of the cells with the one or more nucleic acids is increased by at least 5%, at least 10%, at least 15%, at least 20%, at least 25%, at least 30%, at least 35%, at least 35%, at least 40%, at least 45%, or at least 50% as compared to the transfection of the cells performed under the same conditions but in the absence of the stabilizer.
E83. The method as set forth in any one of E1-E81, wherein the amount of rAAV vector isolated/purified from the transfected cells is increased by at least 5%, at least 10%, at least 15%, at least 20%, at least 25%, at least 30%, at least 35%, at least 35%, at least 40%, at least 45%, or at least 50% or greater as compared to the amount of rAAV vector isolated/purified from the transfected cells under the same conditions but in the absence of the stabilizer.
E84. The method as set forth in any one of E1-E81, wherein the amount of rAAV vector isolated/purified from the transfected cells is increased by at least 2-fold, at least 4-fold, at least 10-fold, at least 20-fold, at least 40-fold, at least 50-fold, at least 60-fold, at least 80-fold, at least 100-fold or greater as compared to the amount of rAAV vector isolated/purified from the transfected cells under the same conditions but in the absence of the stabilizer.
E85. The method as set forth in any one of E1-E81, wherein the rAAV titer is increased by at least 2-fold at least 4-fold, at least 10-fold, at least 20-fold, at least 40-fold, at least 50-fold, at least 60-fold, at least 80-fold, at least 100-fold or greater as compared to the rAAV titer produced under the same conditions but in the absence of the stabilizer.
E86. The method as set forth in any one of E1-E81, wherein the rAAV titer is increased by at least 5%, at least 10%, at least 15%, at least 20%, at least 25%, at least 30%, at least 35%, at least 35%, at least 40%, at least 45%, or at least 50% or greater as compared to the rAAV titer produced under the same conditions but in the absence of the stabilizer.
E87. The method as set forth in any one of E1-E81, wherein the number of rAAV full vectors is increased by at least 5%, at least 10%, at least 15%, at least 20%, at least 25%, at least 30%, at least 35%, at least 35%, at least 40%, at least 45%, or at least 50% or greater as compared to the number of rAAV full vectors produced under the same conditions but in the absence of the stabilizer.
E88. The method as set forth in any one of E1-E81, wherein the number of rAAV empty vectors is decreased by at least 5%, at least 10%, at least 15%, at least 20%, at least 25%, at least 30%, at least 35%, at least 35%, at least 40%, at least 45%, or at least 50% or greater as compared to the number of rAAV empty vectors produced under the same conditions but in the absence of the stabilizer.
E89. The methods as set forth in any one of E1-E81, wherein the rAAV vectors isolated and/or purified in the presence of the stabilizer have a 260:280 ratio of about 1.0 to about 1.2, while the rAAV vectors isolated and/or purified under the same conditions but in the absence of the stabilizer have a 260:280 ratio of about 0.6.
E90. The method as set forth in any one of E1-E89, wherein the cells comprise mammalian cells, yeast cells or insect cells.
E91. The method as set forth in E90, wherein the cells are human embryonic kidney (HEK), Chinese hamster ovary (CHO) cells or insect-derived Sf9 cells.
E92. The method as set forth in E91, wherein the cells comprise Human Embryonic Kidney (HEK) 293 cells.
E93. The method as set forth in E92, wherein the cells are HEK 293E, HEK 293F or HEK 293T cells.
E94. The method as set forth in E92, wherein the HEK 293 cells are adapted for serum-free growth in suspension.
E95. The method as set forth in any one of E1-E94, wherein the cells are stably or transiently transfected.
E96. The method as set forth in any one of E1-E95, wherein the cells are in suspension culture.
E97. The method as set forth in any one of E1-E95, wherein the cells are adherent.
E98. The method as set forth in any one of E1-E97, wherein the cells are grown or maintained in a serum-free culture medium.
E99. The method as set forth in any one of E1-E98, wherein the cells are grown or maintained in roller bottles or expanded roller bottles.
E100. The method as set forth in any one of E1-E98, wherein the cells are grown in bioreactors. 25 E101. The method as set forth in any one of E1-E98, wherein the cells are grown in bags or flasks.
E102. The method as set forth in E100, wherein the cells are grown in a WAVE bioreactor. 30 E103. The method as set forth in E100, wherein the cells are grown in a stirred tank bioreactor.
E104. A composition comprising one or more cationic polymers and a stabilizer.
E105. The composition as set forth in E104, further comprising one or more nucleic acids.
E106. The composition as set forth in E105, further comprising cells.
E107. A composition comprising a stabilizer and one or more nucleic acids.
E108. The composition as set forth in E107, further comprising one or more cationic polymers.
E109. The composition as set forth in E108, further comprising cells.
E110. A composition comprising one or more cationic polymers, a stabilizer, one or more nucleic acids, and cells.
E111. The composition as set forth in any one of E104-E106, E108-E110, wherein the amount of one or more cationic polymers is about 0.01 μg, about 0.02 μg, about 0.04 μg, about 0.06 μg, about 0.08 μg, about 1.0 μg, about 1.2 μg, about 1.4 μg, about 1.6 μg, about 1.8 μg, about 2.0 μg, about 2.2 μg, about 2.4 μg, about 2.6 μg, about 2.8 μg, about 3.0 μg, about 3.2 μg, about 3.4 μg, about 3.6 μg, about 3.8 μg, about 4.0 μg, about 4.2 μg, about 4.6 μg, about 4.8 μg, about 5.0 μg, about 5.5 μg, about 6.0 μg, about 6.5 μg, about 7 μg, about 7.5 μg, about 8.0 μg, about 8.5 μg, about 9.0 μg, about 9.5 μg, about 10 μg, about 12 μg, about 14 μg, about 16 μg, about 18 μg, about 20 μg, about 40 μg, about 60 μg, about 80 μg, about 100 μg, about 0.1 mg, about 1 mg, about 2 mg, about 4 mg, about 6 mg, about 8 mg or about 10 mg.
E112. The composition as set forth in E111, wherein the amount of one or more cationic polymers is about 20 μg, about 22 μg, about 24 μg, about 26 μg, about 26.4 μg, about 28 μg, about 30 μg, about 32 μg, about 34 μg, about 36 μg, about 38 μg, about 40 μg, about 42 μg, about 44 μg, about 46 μg, about 48 μg, about 50 μg, about 52 μg, about 52.8 μg, about 54 μg, about 56 μg, about 58 μg, or about 60 μg.
E113. The composition as set forth in any one or E104-E112, wherein the amount of stabilizer is about 0.01 μg, about 0.02 μg, about 0.04 μg, about 0.06 μg, about 0.08 μg, about 1.0 μg, about 1.2 μg, about 1.4 μg, about 1.6 μg, about 1.8 μg, about 2.0 μg, about 2.2 μg, about 2.4 μg, about 2.6 μg, about 2.8 μg, about 3.0 μg, about 3.2 μg, about 3.4 μg, about 3.6 μg, about 3.8 μg, about 4.0 μg, about 4.2 μg, about 4.6 μg, about 4.8 μg, about 5.0 μg, about 5.5 μg, about 6.0 μg, about 6.5 μg, about 7 μg, about 7.5 μg, about 8.0 μg, about 8.5 μg, about 9.0 μg, about 9.5 μg, about 10 μg, about 12 μg, about 14 μg, about 16 μg, about 18 μg, about 20 μg, about 40 μg, about 60 μg, about 80 μg, about 100 μg, about 0.1 mg, about 1 mg, about 2 mg, about 4 mg, about 6 mg, about 8 mg or about 10 mg.
E114. The composition as set forth in E113, wherein the amount of stabilizer is about 1.0 μg, about 1.2 μg, about 1.4 μg, about 1.6 μg, about 1.8 μg, about 2.0 μg, about 2.2 μg, about 2.4 μg, about 2.6 μg, about 2.64 μg, about 2.8 μg, about 3.0 μg, about 3.2 μg, about 3.4 μg, about 3.6 μg, about 3.8 μg, about 4.0 μg, about 4.2 μg, about 4.6 μg, about 4.8 μg, about 5.0 μg, about 5.5 μg, about 6.0 μg, about 6.5 μg, about 7 μg, about 7.5 μg, about 8.0 μg, about 8.5 μg, about 9.0 μg, about 9.5 μg, about 10 μg, about 12 μg, about 13 μg, about 14 μg, about 16 μg, about 18 μg or about 20 μg.
E115. The composition as set forth in any one of E105-E114, wherein the amount of one or more nucleic acids is about 0.01 μg, about 0.02 μg, about 0.04 μg, about 0.06 μg, about 0.08 μg, about 1.0 μg, about 1.2 μg, about 1.4 μg, about 1.6 μg, about 1.8 μg, about 2.0 μg, about 2.2 μg, about 2.4 μg, about 2.6 μg, about 2.8 μg, about 3.0 μg, about 3.2 μg, about 3.4 μg, about 3.6 μg, about 3.8 μg, about 4.0 μg, about 4.2 μg, about 4.6 μg, about 4.8 μg, about 5.0 μg, about 5.5 μg, about 6.0 μg, about 6.5 μg, about 7 μg, about 7.5 μg, about 8.0 μg, about 8.5 μg, about 9.0 μg, about 9.5 μg, about 10 μg, about 12 μg, about 14 μg, about 16 μg, about 18 μg, about 20 μg, about 40 μg, about 60 μg, about 80 μg, about 100 μg, about 0.1 mg, about 1 mg, about 2 mg, about 4 mg, about 6 mg, about 8 mg or about 10 mg.
E116. The composition as set forth in E115, wherein the amount of one or more nucleic acids is about 12 μg or 24 μg.
E117. The composition as set forth in any one of E106, E109-E116, wherein the cells are at a cell density of at least 1×10⁵ cells/mL, at least 2×10⁵ cells/mL, at least 4×10⁵ cells/mL, at least 6×10⁵ cells/mL, at least 8×10⁵ cells/mL, at least 0.5×10⁶ cells/mL, at least 1×10⁶ cells/mL, at least 2×10⁶ cells/mL, at least 4×10⁶ cells/mL, at least 6×10⁶ cells/mL, at least 8×10⁶ cells/mL, at least 10×10⁶ cells/mL, at least 12×10⁶ cells/mL, at least 14×10⁶ cells/mL, at least 16×10⁶ cells/mL, at least 18×10⁶ cells/mL, at least 20×10⁶ cells/mL, at least 22×10⁶ cells/mL, at least 24×10⁶ cells/mL, at least 26×10⁶ cells/mL, at least 28×10⁶ cells/mL, at least 30×10⁶ cells/mL, at least 32×10⁶ cells/mL, at least 34×10⁶ cells/mL, at least 36×10⁶ cells/mL, at least 38×10⁶ cells/mL, at least 40×10⁶ cells/mL, at least 42×10⁶ cells/mL, at least 44×10⁶ cells/mL, at least 46×10⁶ cells/mL, at least 48×10⁶ cells/mL, at least 50×10⁶ cells/mL, or at least 52×10⁶ cells/mL.
E118. The composition as set forth in E117, wherein the cells are at a density of at least 12×10⁶ cells/mL or at least 24×10⁶ cells/mL.
E119. A composition comprising about 12×10⁶ cells/mL, about 2.2 μg per million cells of one or more cationic polymers, about 10% of stabilizer relative to the one or more cationic polymers, and about 1 μg per million cells of one or more nucleic acids.
E120. A composition comprising about 24×10⁶ cells/mL, about 2.2 μg per million cells of one or more cationic polymers, about 25% of stabilizer relative to the one or more cationic polymers, and about 1 μg per million cells of one or more nucleic acids.

### DESCRIPTION OF DRAWINGS

FIG. 1 depicts the structure comparison of positively charged PEI with neutrally charged PEG.

FIG. 2 shows the turbidity profile of transfection cocktail prepared with and without stabilizer.

FIG. 3 depicts a dynamic light scattering (DLS) based experiment showing the decrease in average particle size with increasing amount of stabilizer in the transfection cocktail.

FIG. 4 shows the comparison of cell viability 24, 48 and 72 hrs post transfection with increasing percentage of stabilizer (relative to PEI) in the transfection cocktail.

FIG. 5 shows AAV titer data (vg/ml) measured using digital droplet polymerase chain reaction (ddPCR). Y axis showing the AAV titer (vg/ml) and X-axis shows increased amount of stabilizer (relative to the PEI) in the transfection cocktal. The titer is adjusted for dilution post transfection.

FIG. 6 shows the comparison of cell viability 24, 48 and 72 hrs post transfection with increasing percentage of stabilizer (branched and linear) relative to PEI.

FIG. 7 shows AAV titer data (vg/ml) measured using ddPCR. Y axis showing the AAV titer (vg/ml) and X-axis shows increased amounts of stabilizers (branched vs linear) relative to the PEI. The titer is adjusted for dilution post transfection.

FIG. 8 shows the comparison of cell viability 24, 48 and 72 hrs post transfection with increasing percentage of stabilizer (containing 15 versus 55 PEG per bPEI) relative to PEI.

FIG. 9 shows AAV titer data (vg/ml) measured using ddPCR. Y axis showing the AAV titer (vg/ml) and X-axis shows increased amounts of stabilizers (containing 15 versus 55 PEG per bPEI) relative to the PEI. The titer is adjusted for dilution post transfection.

FIG. 10 shows the comparison of AAV titers with increasing transfection cocktail incubation time. The transfection cocktails (PEI-DNA) were prepared with and without stabilizer (bPEI25k.PEG5k).

FIG. 11 depicts the corresponding 260:280 ratio graph to FIG. 9.

FIG. 12 shows AAV titer data (vg/ml) measured using ddPCR. Y axis showing the AAV titer (vg/ml) and X-axis shows increased amounts of stabilizers (bPEi15k.PEG5k) and PEG (PEG35k). The bars in the first two columns are a positive transfection control, wherein the transfection cocktail was prepared with plasmid DNA and PEI alone (i.e., no stabilizer) and then incubated for 2 minutes before addition to the cell culture. The titer is adjusted for dilution post transfection. The experiments were conducted separately for PEG35k and bPEI25k.PEG5k. The graphs have been plotted together to compare the two experiments.

FIG. 13 shows AAV titer data (vg/ml) measured using ddPCR. Y axis depicts the AAV titer (vg/ml), while X-axis shows transfections performed using transfection cocktails containing either 2.2 ug PEI alone (i.e., no stabilizer) (bars 1 and 2), 2.2 ug PEI with 0.22 ug of stabilizer (bars 3 and 4), 2.42 ug of stabilizer alone (i.e., no PEI) (bars 5 and 6), 11 ug of stabilizer alone (i.e., no PEI) (bars 7 and 8), or 4 ug PEI with 0.4 ug stabilizer (bars 9 and 10). The various transfection cocktails were prepared and incubated for either 2 minutes or 60 minutes before addition to the cell culture.

FIG. 14 shows AAV titer data (vg/ml) for 2 liter bioreactor runs using transfection stabilizer measured using ddPCR. Y axis shows the AAV titer (vg/ml) and X-axis shows increased amount of transfection cocktail incubation time. Sample with previously determined titer was used as a positive control for ddPCR assay.

FIG. 15 shows AAV titer data (vg/ml) measured using ddPCR. Y axis shows the AAV titer (vg/ml) and X-axis shows increased amount of transfection cocktail incubation time. The titer is adjusted for dilution post transfection. Sample with previously determined titer was used as a positive control for ddPCR assay.

FIG. 16 shows the comparison of cell viability 24, 48 and 72 hrs post transfection with increasing percentage of stabilizer (pLL.PEG_1 vs pLL.PEG_2) relative to PEI.

FIG. 17 shows AAV titer data (vg/ml) using another transfection stabilizer compound measured using ddPCR.

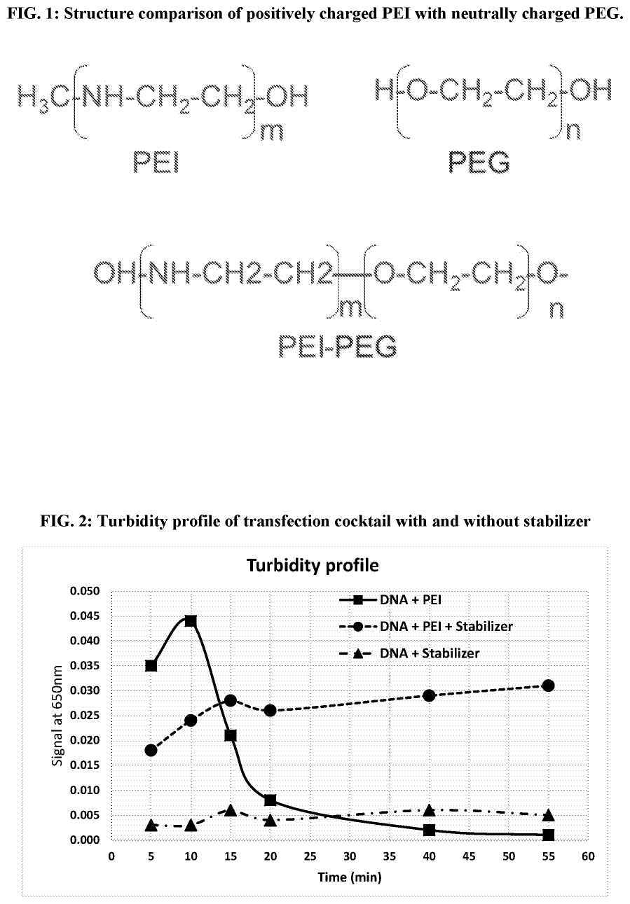

### DETAILED DESCRIPTION

Cationic polymers, such as polyethylenimine (PEI), are simple, inexpensive and effective reagents for transfecting cells. According to the classic protocol for transfection, PEI and DNA are pre-mixed and incubated for some time to form polyplexes before transfection (“transfection cocktail incubation time”). However, these polyplexes are unstable and depending on the length of the transfection cocktail incubation time, the DNA-PEI complexes can aggregate into large particles which undermines their ability to transfect cells. In fact, irrespective of the transfection cocktail incubation time, any time lag between the preparation of the transfection cocktail and contacting cells for transfection increases the possibility of DNA-PEI complex aggregation. Without being bound by theory, it is hypothesized that the rate of aggregation also depends on temperature, mixing conditions, cell density, and/or DNA concentration. Higher temperatures, strong mixing conditions, higher cell densities and DNA concentrations may contribute to faster aggregation of the DNA-PEI complexes. This severely compromises the ability to use cationic polymers for transfecting cells to produce, for example, AAV vectors, on a large scale.

Disclosed herein are compositions and methods for stabilizing a transfection cocktail containing DNA-cationic polymer complexes for an extended time, while maintaining high transfection efficiency. Such stabilized transfection cocktail can be used to generate transfected cells that can produce, for example, rAAV vectors on a large scale without impacting the key attributes of the virus production, such as, titer, DNA packaged rAAV particle fraction, and rAAV vector purification profile. In particular, the inventors were able to stabilize the DNA-PEI complexes (i.e., prevent aggregation into large particles) for at least 8 hours (see Example 9), while obtaining comparable rAAV titers (see, for example, Examples 3-10). In addition, the inventors determined the optimal concentration and the order of addition of stabilizer in the transfection cocktail required to produce maximal rAAV titer yield for both high and low cell density transfections.

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999).

### Definitions

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids and polynucleotides include genomic DNA, CDNA and antisense DNA, plasmid, and spliced or unspliced mRNA, rRNA tRNA and inhibitory DNA or RNA (RNAi, e.g., small or short hairpin (sh)RNA, microRNA (miRNA), small or short interfering (si)RNA, trans-splicing RNA, or antisense RNA). Nucleic acids and polynucleotides include naturally occurring, synthetic, and intentionally modified or altered sequences (e.g., variant nucleic acid).

A “plasmid” is a form of nucleic acid or polynucleotide that typically has additional elements for expression (e.g., transcription, replication, etc.) or propagation (replication) of the plasmid. A plasmid as used herein also can be used to reference nucleic acid and polynucleotide sequences. Accordingly, in all aspects the invention compositions and methods are applicable to plasmids, nucleic acids and polynucleotides, e.g., for introducing plasmids, nucleic acid or polynucleotide into cells, for transducing (transfecting) cells with plasmid, nucleic acid or polynucleotide, for producing transduced (transfected) cells that have a plasmid, nucleic acid or polynucleotide, to produce cells that produce viral (e.g., AAV) vectors, to produce viral (e.g., AAV) vectors, to produce cell culture medium that has viral (e.g., AAV) vectors, etc.

In some embodiments, a nucleic acid or plasmid refers to a sequence which encodes a protein. Such proteins can be wild-type or a variant, modified or chimeric protein. A ‘variant protein’ can mean a modified protein such that the modified protein has an amino acid alteration compared to wild-type protein.

Proteins encoded by a nucleic acid or plasmid include therapeutic proteins. Non-limiting examples include a blood clotting factor (e.g., Factor XIII, Factor IX, Factor X, Factor VIII, Factor VIIa, or protein C), mini-dystrophin, C1 esterase inhibitor, copper transporting P-type ATPase (ATP7B), copper-zinc superoxide dismutase 1 (SOD1), apoE2, arginino succinate synthase, acid alpha-glucosidase, β-Glucocerebrosidase, a-galactosidase, CI inhibitor serine protease inhibitor, CFTR (cystic fibrosis transmembrane regulator protein), an antibody, retinal pigment epithelium-specific 65 kDa protein (RPE65), erythropoietin, LDL receptor, lipoprotein lipase, ornithine transcarbamylase, β-globin, a-globin, spectrin, a-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor (e.g., insulin-like growth factors 1 and 2, platelet derived growth factor, epidermal growth factor, nerve growth factor, neurotrophic factor-3 and -4, brain-derived neurotrophic factor, glial derived growth factor, transforming growth factor a and β, etc.), a cytokine (e.g., a-interferon, β-interferon, interferon-γ, interleukin-2, interleukin-4, interleukin 12, granulocyte-macrophage colony stimulating factor, lymphotoxin, etc.), a suicide gene product (e.g., herpes simplex virus thymidine kinase, cytosine deaminase, diphtheria toxin, cytochrome P450, deoxycytidine kinase, tumor necrosis factor, etc.), a drug resistance protein (e.g., that provides resistance to a drug used in cancer therapy), a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitopes, or hCDR1, insulin, glucokinase, guanylate cyclase 2D (LCA-GUCY2D), Rab escort protein 1 (Choroideremia), LCA 5 (LCA-Lebercilin), ornithine ketoacid aminotransferase (Gyrate Atrophy), Retinoschisin 1 (X-linked Retinoschisis), USH1C (Usher's Syndrome 1C), X-linked retinitis pigmentosa GTPase (XLRP), MERTK (AR forms of RP: retinitis pigmentosa), DFNB 1 (Connexin 26 deafness), ACHM 2, 3 and 4 (Achromatopsia), PKD-1 or PKD-2 (Polycystic kidney disease), gene deficiencies causative of lysosomal storage diseases (e.g., sulfatases, N-acetylglucosamine-l-phosphate transferase, cathepsin A, GM2-AP, NPC1, VPC2, Sphingolipid activator proteins, etc.), one or more zinc finger nucleases for genome editing, or donor sequences used as repair templates for genome editing.

In some embodiments, a nucleic acid or plasmid refers to a sequence which produces a transcript when transcribed. Such transcripts can be RNA, such as inhibitory RNA (RNAi, e.g., small or short hairpin (sh)RNA, microRNA (miRNA), small or short interfering (si)RNA, trans-splicing RNA, or antisense RNA).

Non-limiting examples include inhibitory nucleic acids that inhibit expression of: huntingtin (HTT) gene, a gene associated with dentatorubropallidolusyan atropy (e.g., atrophin 1, ATN1); androgen receptor on the X chromosome in spinobulbar muscular atrophy, human Ataxin-1, -2, -3, and -7, Cav2.1 P/Q voltage-dependent calcium channel is encoded by the (CACNA1A), TATA-binding protein, Ataxin 8 opposite strand, also known as ATXN80S, Serine/threonine-protein phosphatase 2A 55 kDa regulatory subunit B beta isoform in spinocerebellar ataxia (type 1, 2, 3, 6, 7, 8, 12 17), FMR1 (fragile X mental retardation 1) in fragile X syndrome, FMR1 (fragile X mental retardation 1) in fragile X-associated tremor/ataxia syndrome, FMR1 (fragile X mental retardation 2) or AF4/FMR2 family member 2 in fragile XE mental retardation; Myotonin-protein kinase (MT-PK) in myotonic dystrophy; Frataxin in Friedreich's ataxia; a mutant of superoxide dismutase 1 (SOD1) gene in amyotrophic lateral sclerosis; a gene involved in pathogenesis of Parkinson's disease and/or Alzheimer's disease; apolipoprotein B (APOB) and proprotein convertase subtilisin/kexin type 9 (PCSK9), hypercoloesterolemia; HIV Tat, human immunodeficiency virus transactivator of transcription gene, in HIV infection; HIV TAR, HIV TAR, human immunodeficiency virus transactivator response element gene, in HIV infection; C-C chemokine receptor (CCR5) in HIV infection; Rous sarcoma virus (RSV) nucleocapsid protein in RSV infection, liver-specific microRNA (miR-122) in hepatitis C virus infection; p53, acute kidney injury or delayed graft function kidney transplant or kidney injury acute renal failure; protein kinase N3 (PKN3) in advance recurrent or metastatic solid malignancies; LMP2, LMP2 also known as proteasome subunit beta-type 9 (PSMB 9), metastatic melanoma; LMP7, also known as proteasome subunit beta-type 8 (PSMB 8), metastatic melanoma; MECL1 also known as proteasome subunit beta-type 10 (PSMB 10), metastatic melanoma; vascular endothelial growth factor (VEGF) in solid tumors; kinesin spindle protein in solid tumors, apoptosis suppressor B-cell CLL/lymphoma (BCL-2) in chronic myeloid leukemia; ribonucleotide reductase M2 (RRM2) in solid tumors; Furin in solid tumors; polo-like kinase 1 (PLK1) in liver tumors, diacylglycerol acyltransferase 1 (DGAT1) in hepatitis C infection, beta-catenin in familial adenomatous polyposis; beta2 adrenergic receptor, glaucoma; RTP801/Reddl also known as DAN damage-inducible transcript 4 protein, in diabetic macular oedma (DME) or age-related macular degeneration; vascular endothelial growth factor receptor I (VEGFR1) in age-related macular degeneration or choroidal neovascularization, caspase 2 in non-arteritic ischaemic optic neuropathy; Keratin 6A N17K mutant protein in pachyonychia congenital; influenza A virus genome/gene sequences in influenza infection; severe acute respiratory syndrome (SARS) coronavirus genome/gene sequences in SARS infection; respiratory syncytial virus genome/gene sequences in respiratory syncytial virus infection; Ebola filovirus genome/gene sequence in Ebola infection; hepatitis B and C virus genome/gene sequences in hepatitis B and C infection; herpes simplex virus (HSV) genome/gene sequences in HSV infection, coxsackievirus B3 genome/gene sequences in coxsackievirus B3 infection; silencing of a pathogenic allele of a gene (allele-specific silencing) like torsin A (TOR1A) in primary dystonia, pan-class I and HLA-allele specific in transplant; mutant rhodopsin gene (RHO) in autosomal dominantly inherited retinitis pigmentosa (adRP); or the inhibitory nucleic acid binds to a transcript of any of the foregoing genes or sequences.

Nucleic acids (plasmids) can be single, double, or triplex, linear or circular, and can be of any length. In discussing nucleic acids (plasmids), a sequence or structure of a particular polynucleotide may be described herein according to the convention of providing the sequence in the 5′ to 3′ direction.

Cells contacted with the transfection cocktail as set forth herein may be referred to as “host cells”. A “host cell” denotes, for example, microorganisms, yeast cells, insect cells, and mammalian cells, that can be, or have been, used as recipients of nucleic acid (plasmid) encoding packaging proteins, such as AAV packaging proteins, a nucleic acid (plasmid) encoding helper proteins, a nucleic acid (plasmid) that encodes a protein or is transcribed into a transcript of interest, or other transfer nucleic acid (plasmid). The term includes the progeny of the original cell, which has been transduced or transfected. Thus, a “host cell” as used herein generally refers to a cell which has been transduced or transfected with an exogenous nucleic acid sequence. It is understood that the progeny of a single parental cell may not necessarily be completely identical in morphology or in genomic or total nucleic acid complement as the original parent, due to natural, accidental, or deliberate mutation.

The terms “transduce” and “transfect” refer to introduction of a molecule such as a nucleic acid (plasmid) into a host cell. A cell has been “transduced” or “transfected” when exogenous nucleic acid has been introduced inside the cell membrane. Accordingly, a “transduced cell” or a “transfected cell” is a cell into which a nucleic acid or polynucleotide has been introduced, or a progeny thereof in which an exogenous nucleic acid has been introduced.

In a “transduced” or “transfected” cell, the nucleic acid (plasmid) may or may not be integrated into genomic nucleic acid of the recipient cell. If an introduced nucleic acid becomes integrated into the nucleic acid (genomic DNA) of the recipient cell or organism it can be stably maintained in that cell or organism and further passed on to or inherited by progeny cells or organisms of the recipient cell or organism. Finally, the introduced nucleic acid may exist in the recipient cell or host organism extrachromosomally, or only transiently.

The term “vector” refers to small carrier nucleic acid molecule, a plasmid, virus (e.g., AAV vector), or other vehicle that can be manipulated by insertion or incorporation of a nucleic acid. Such vectors can be used for genetic manipulation (i.e., “cloning vectors”), to introduce/transfer polynucleotides into cells, and to transcribe or translate the inserted polynucleotide in cells.

An “expression vector” is a specialized vector that contains a gene or nucleic acid sequence with the necessary regulatory regions needed for expression in a host cell. A vector nucleic acid sequence generally contains at least an origin of replication for propagation in a cell and optionally additional elements, such as a heterologous polynucleotide sequence, expression control element (e.g., a promoter, enhancer), intron, ITR(s), selectable marker (e.g., antibiotic resistance), polyadenylation signal. For purposes of the invention, a “vector” as set forth herein is within the scope of a “plasmid” as this term is used herein.

A viral vector is derived from or based upon one or more nucleic acid elements that comprise a viral genome. Particular viral vectors include lentivirus, pseudo-typed lentivirus and parvo-virus vectors, such as adeno-associated virus (AAV) vectors. The AAV vector may comprise any of natural (i.e., wild type) or non-natural (e.g., recombinant) AAV serotypes (e.g., but not limited to AAV1-12), an AAV VP1, VP2 and/or VP3 capsid protein, or a modified or variant AAV VP1, VP2 and/or VP3 capsid protein, or wild-type AAV VP1, VP2 and/or VP3 capsid protein.

The term “recombinant”, as a modifier of vector, such as recombinant viral, e.g., lenti- or parvo-virus (e.g., AAV) vectors, as well as a modifier of sequences such as recombinant polynucleotides and polypeptides, means that the compositions have been manipulated (i.e., engineered) in a fashion that generally does not occur in nature. A particular example of a recombinant vector, such as an AAV vector would be where a polynucleotide that is not normally present in the wild-type viral (e.g., AAV) genome is inserted within the viral genome, i.e., is heterologous. Although the term “recombinant” is not always used herein in reference to vectors, such as viral and AAV vectors, as well as sequences such as polynucleotides, recombinant forms including polynucleotides, are expressly included in spite of any such omission.

A recombinant viral vector or AAV vector is derived from the wild type genome of a virus, such as AAV by using molecular methods to remove the wild type genome from the virus (e.g., AAV), and replacing with a non-native nucleic acid, such as a nucleic acid transcribed into a transcript or that encodes a protein. Typically, for AAV one or both inverted terminal repeat (ITR) sequences of AAV genome are retained in the AAV vector. A recombinant viral vector (e.g., AAV) is distinguished from a viral (e.g., AAV) genome, since all or a part of the viral genome has been replaced with a non-native (i.e., heterologous) sequence with respect to the viral (e.g., AAV) genomic nucleic acid. Incorporation of a non-native sequence therefore defines the viral vector (e.g., AAV) as a recombinant vector, which in the case of AAV can be referred to as a rAAV vector.

A recombinant vector (e.g., lenti-, parvo-, AAV) sequence can be packaged—referred to herein as a particle for subsequent infection (transduction) of a cell, ex vivo, in vitro or in vivo. Where a recombinant vector sequence is encapsidated or packaged into an AAV particle, the particle can also be referred to as a rAAV. Such particles include proteins that encapsidate or package the vector genome. Particular examples include viral envelope proteins, and in the case of AAV, capsid proteins, such as AAV VP1, VP2 and VP3.

A vector genome refers to the portion of the recombinant plasmid sequence that is ultimately packaged or encapsidated to form a viral (e.g., AAV) particle. In cases where recombinant plasmids are used to construct or manufacture recombinant vectors, the vector genome does not include the portion of the plasmid that does not correspond to the vector genome sequence of the recombinant plasmid. This non vector genome portion of the recombinant plasmid is referred to as the plasmid backbone, which is important for cloning and amplification of the plasmid, a process that is needed for propagation and recombinant virus production, but is not itself packaged or encapsidated into virus (e.g., AAV) particles. Thus, a vector genome refers to the nucleic acid that is packaged or encapsidated by virus (e.g., AAV).

The terms “empty capsid” and “empty particle”, refer to an AAV virion that includes an AAV protein shell but that lacks in whole or part a nucleic acid that encodes a protein or is transcribed into a transcript of interest flanked by AAV ITRs. Accordingly, the empty capsid does not function to transfer a nucleic acid that encodes a protein or is transcribed into a transcript of interest into the host cell. However, empty capsid formulations have utility in other applications.

As used herein, AAV packaging proteins refer to AAV-derived sequences which function in trans for productive AAV replication. Thus, AAV packaging proteins are encoded by the major AAV open reading frames (ORFs), rep and cap. The rep proteins have been shown to possess many functions, including, among others: recognition, binding and nicking of the AAV origin of DNA replication; DNA helicase activity; and modulation of transcription from AAV (or other heterologous) promoters. The cap (capsid) proteins supply necessary packaging functions. AAV packaging proteins are used herein to complement AAV functions in trans that are missing from AAV vectors.

The nucleic acids encoding AAV packaging proteins refer generally to a nucleic acid molecule that includes nucleotide sequences providing AAV functions deleted from an AAV vector which is to be used to produce a transducing recombinant AAV vector. The nucleic acids encoding AAV packaging proteins are commonly used to provide transient expression of AAV rep and/or cap genes to complement missing AAV functions that are necessary for AAV replication; however, the nucleic acid constructs lack AAV ITRs and can neither replicate nor package themselves. Nucleic acids encoding AAV packaging proteins can be in the form of a plasmid, phage, transposon, cosmid, virus, or virion. A number of nucleic acid constructs have been described, such as the commonly used plasmids pAAV/Ad and pIM29+45 which encode both Rep and Cap expression products. See, e.g., Samulski et al. (1989) J. Virol. 63:3822-3828; and McCarty et al. (1991) J. Virol. 65:2936-2945. A number of vectors have been described which encode Rep and/or Cap expression products (e.g., U.S. Pat. Nos. 5,139,941 and 6,376,237).

The term nucleic acids encoding helper proteins refers generally to a nucleic acid molecule(s) that includes nucleotide sequences encoding proteins that provide helper function(s). A vector with nucleic acid(s) encoding helper protein(s) can be transfected into a suitable host cell, wherein the vector is then capable of supporting AAV virion production in the host cell. Expressly excluded from the term are infectious viral particles, as they exist in nature, such as adenovirus, herpesvirus or vaccinia virus particles.

Helper protein vectors can be in the form of a plasmid, phage, transposon or cosmid. In particular, it has been demonstrated that the full-complement of adenovirus genes are not required for helper functions. For example, adenovirus mutants incapable of DNA replication and late gene synthesis have been shown to be permissive for AAV replication. Ito et al., (1970) J. Gen. Virol. 9:243; Ishibashi et al, (1971) Virology 45:317.

Mutants within the E2B and E3 regions have been shown to support AAV replication, indicating that the E2B and E3 regions are probably not involved in providing helper function. Carter et al:, (1983) Virology 126:505. However, adenoviruses defective in the E1 region, or having a deleted E4 region, are unable to support AAV replication. Thus, for adenoviral helper proteins, EIA and E4 regions are likely required for AAV replication, either directly or indirectly. Laughlin et al., (1982) J. Virol. 41:868; Janik et al., (1981) Proc. Natl. Acad. Sci. USA 78: 1925; Carter et al., (1983) Virology 126:505. Other characterized Ad mutants include: EIB (Laughlin et al. (1982), supra; Janik et al. (1981), supra; Ostrove et al., (1980) Virology 104:502); E2A (Handa et al., (1975) J. Gen. Virol. 29:239; Strauss et al., (1976) J. Virol. 17: 140; Myers et al., (1980) J. Virol. 35:665; Jay et al., (1981) Proc. Natl. Acad. Sci. USA 78:2927; Myers et al., (1981) J. Biol. Chem. 256:567); E2B (Carter, Adeno-Associated Virus Helper Functions, in I CRC Handbook of Parvoviruses (P. Tijssen ed., 1990)); E3 (Carter et al. (1983), supra); and E4 (Carter et al. (1983), supra; Carter (1995)).

Studies of the helper proteins provided by adenoviruses having mutations in the EIB have reported that El B55k is required for AAV virion production, while EIB 19k is not. In addition, International Publication WO 97/17458 and Matshushita et al., (1998) Gene Therapy 5:938-945, describe helper function vectors encoding various Ad genes. An example of a helper vector comprise an adenovirus VA RNA coding region, an adenovirus E4 ORF6 coding region, an adenovirus E2A 72 kD coding region, an adenovirus EIA coding region, and an adenovirus EIB region lacking an intact E I BS5k coding region (see, e.g., International Publication No. WO 01/83797).

A transgene is used herein to conveniently refer to a nucleic acid that is intended or has been introduced into a cell or organism. Transgenes include any nucleic acid, such as a gene that is transcribed into a transcript or that encodes a polypeptide or protein of interest.

An expression control element refers to nucleic acid sequence(s) that influence expression of an operably linked nucleic acid. Control elements, including expression control elements as set forth herein such as promoters, enhancers, etc. Vector sequences including AAV vectors can include one or more expression control elements. Typically, such elements are included to facilitate proper heterologous polynucleotide transcription and if appropriate translation (e.g., a promoter, enhancer, splicing signal for introns, maintenance of the correct reading frame of the gene to permit in-frame translation of mRNA and, stop codons etc.). Such elements typically act in cis, referred to as a “cis acting” element, but may also act in trans.

Expression control can be at the level of transcription, translation, splicing, message stability, etc. Typically, an expression control element that modulates transcription is juxtaposed near the 5′ end (i.e., upstream) of a transcribed nucleic acid. Expression control elements can also be located at the 3′ end (i.e., downstream) of the transcribed sequence or within the transcript (e.g., in an intron). Expression control elements can be located adjacent to or at a distance away from the transcribed sequence (e.g., 1-10, 10-25, 25-50, 50-100, 100 to 500, or more nucleotides from the polynucleotide), even at considerable distances. Nevertheless, owing to the length limitations of certain vectors, such as AAV vectors, expression control elements will typically be within 1 to 1000 nucleotides from the transcribed nucleic acid.

Functionally, expression of operably linked nucleic acid is at least in part controllable by the element (e.g., promoter) such that the element modulates transcription of the nucleic acid and, as appropriate, translation of the transcript. A specific example of an expression control element is a promoter, which is usually located 5′ of the transcribed sequence. A promoter typically increases an amount expressed from operably linked nucleic acid as compared to an amount expressed when no promoter exists.

An enhancer as used herein can refer to a sequence that is located adjacent to the heterologous polynucleotide. Enhancer elements are typically located upstream of a promoter element but also function and can be located downstream of or within a nucleic acid sequence. Hence, an enhancer element can be located 100 base pairs, 200 base pairs, or 300 or more base pairs upstream or downstream of a nucleic acid. Enhancer elements typically increase expressed of an operably linked nucleic acid above expression afforded by a promoter element.

An expression construct may comprise regulatory elements which serve to drive expression in a particular cell or tissue type. Expression control elements (e.g., promoters) include those active in a particular tissue or cell type, referred to herein as a “tissue-specific expression control elements/promoters.” Tissue-specific expression control elements are typically active in specific cell or tissue (e.g., liver). Expression control elements are typically active in particular cells, tissues or organs because they are recognized by transcriptional activator proteins, or other regulators of transcription, that are unique to a specific cell, tissue or organ type. Such regulatory elements are known to those of skill in the art (see, e.g., Sambrook et al. (1989) and Ausubel et al. (1992)). [0173] The incorporation of tissue specific regulatory elements in the plasmids of the invention provides for at least partial tissue tropism for expression of the nucleic acid. Examples of promoters that are active in liver are the TTR promoter (e.g. mutant TTR promoter), human alpha 1-antitrypsin (hAAT) promoter; albumin, Miyatake, et al. J. Virol., 71:5124-32 (1997); hepatitis B virus core promoter, Sandig, et al., Gene Ther. 3: 1002-9 (1996); alpha-fetoprotein (AFP), Arbuthnot, et al., Hum. Gene. Ther., 7: 1503-14 (1996)], among others. An example of an enhancer active in liver is apolipoprotein E (apoE) HCR-1 and HCR-2 (Allan et al., J. Biol. Chem., 272:29113-19 (1997)).

Expression control elements also include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences and the other viral promoters/enhancers active in a variety of mammalian cell types, or synthetic elements that are not present in nature (see, e.g., Boshart et al, Cell, 41:521-530 (1985)), the SV40 promoter, the dihydrofolate reductase promoter, the cytoplasmic 3-actin promoter and the phosphoglycerol kinase (PGK) promoter.

Additional elements for vectors include, without limitation, a transcription termination signal or stop codon, 5′ or 3′ untranslated regions (e.g., polyadenylation (polyA) sequences) which flank a sequence, such as one or more copies of an AAV ITR sequence, filler or stuffer polynucleotide sequences or an intron.

Filler or stuffer polynucleotide sequences improve packaging and reduce the presence of contaminating nucleic acid. AAV vectors typically accept inserts of DNA having a size range which is generally about 4 kb to about 5.2 kb, or slightly more. Thus, for shorter sequences, inclusion of a stuffer or filler in order to adjust the length to near or at the normal size of the virus genomic sequence acceptable for AAV vector packaging into virus particle. In various embodiments, a filler/stuffer nucleic acid sequence is an untranslated (non-protein encoding) segment of nucleic acid. For a nucleic acid sequence less than 4.7 Kb, the filler or stuffer polynucleotide sequence has a length that when combined (e.g., inserted into a vector) with the sequence has a total length between about 3.0-5.5 Kb, or between about 4.0-5.0 Kb, or between about 4.3-4.8 Kb.

An intron can also function as a filler or stuffer polynucleotide sequence in order to achieve a length for AAV vector packaging into a virus particle. Introns and intron fragments that function as a filler or stuffer polynucleotide sequence also can enhance expression.

The term operably linked means that the regulatory sequences necessary for expression of a coding sequence are placed in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

In the example of an expression control element in operable linkage with a nucleic acid, the relationship is such that the control element modulates expression of the nucleic acid. More specifically, for example, two DNA sequences operably linked means that the two DNAs are arranged (cis or trans) in such a relationship that at least one of the DNA sequences is able to exert a physiological effect upon the other sequence.

The term isolated, when used as a modifier of a composition, means that the compositions are made by the hand of man or are separated, completely or at least in part, from their naturally occurring in vivo environment. Generally, isolated compositions are substantially free of one or more materials with which they normally associate with in nature, for example, one or more contaminants such as protein, nucleic acid, lipid, carbohydrate, cell membrane.

The term isolated does not exclude combinations produced by the hand of man, for example, a recombinant vector (e.g., rAAV) sequence, or virus particle that packages or encapsidates a vector genome and a pharmaceutical formulation. The term “isolated” also does not exclude alternative physical forms of the composition, such as hybrids/chimeras, multimers/oligomers, modifications (e.g., phosphorylation, glycosylation, lipidation) or derivatized forms, or forms expressed in host cells produced by the hand of man.

The term substantially pure refers to a preparation comprising at least 50-60% by weight the compound of interest (e.g., nucleic acid, oligonucleotide, protein, etc.). The preparation can comprise at least 75% by weight, or about 90-99% by weight, of the compound of interest. Purity is measured by methods appropriate for the compound of interest (e.g. chromatographic methods, agarose or polyacrylamide gel electrophoresis, HPLC analysis, and the like).

Reference to about a value or parameter herein includes (and describes) embodiments that are directed to that value or parameter per se. For example, description referring to “about X” includes description of “X.” Numeric ranges are inclusive of the numbers defining the range. Generally speaking, the term “about” refers to the indicated value of the variable and to all values of the variable that are within the experimental error of the indicated value (e.g. within the 95% confidence interval for the mean) or within 10 percent of the indicated value, whichever is greater.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

When introducing elements of the present invention or the preferred embodiment(s) thereof, the articles “a”, “an”, “the” and “said” are intended to mean that there are one or more of the elements. The terms “comprising”, “comprise”, “comprises”, “including” and “having” are intended to be inclusive and mean that there may be additional elements other than the listed elements. It is understood that wherever embodiments are described herein with the language “comprising,” otherwise analogous embodiments described in terms of “consisting of” and/or “consisting essentially of” are also provided.

Unless otherwise defined, all technical and scientific terms used herein have the same meaning as commonly understood by one of ordinary skill in the art to which this invention belongs. In case of conflict, the present specification, including definitions, will control. Unless otherwise required by context, singular terms shall include pluralities and plural terms shall include the singular.

Exemplary methods and materials are described herein, although methods and materials similar or equivalent to those described herein can also be used in the practice or testing of the present invention. The materials, methods, and examples are illustrative only and not intended to be limiting.

### Compositions and Methods

In some aspects, a method for transfecting cells with one or more nucleic acids is provided. The method comprises the following steps: (i) preparing a transfection cocktail comprising one or more cationic polymers, a stabilizer and one or more nucleic acids, (ii) contacting the transfection cocktail prepared in step (i) with cells to be transfected to form a mixture, and (iii) incubating the mixture of step (ii) thereby transfecting the cells with the one or more nucleic acids.

In some aspects, a method for transfecting cells with one or more nucleic acids is provided. The method comprises the following steps: (i) contacting the transfection cocktail with cells to be transfected to form a mixture, and (iii) incubating the mixture of step (ii) thereby transfecting the cells with the one or more nucleic acids, wherein the transfection cocktail comprises one or more cationic polymers, a stabilizer and one or more nucleic acids.

In some aspects, a method for making cells that produce recombinant adeno-associated viral (rAAV) vector is provided. The method comprises the following steps: (i) preparing a transfection cocktail comprising one or more cationic polymers, a stabilizer and one or more nucleic acids, (ii) contacting the transfection cocktail prepared in step (i) with cells to be transfected to form a mixture, and (iii) incubating the mixture of step (ii) thereby making transfected cells that produce rAAV vector.

In some aspects, a method for increasing transfection of cells with one or more nucleic acids is provided. The method comprises the following steps: (i) preparing a transfection cocktail comprising one or more cationic polymers, a stabilizer and one or more nucleic acids, (ii) contacting the transfection cocktail prepared in step (i) with cells to be transfected to form a mixture, and (iii) incubating the mixture of step (ii), whereby transfection of the cells with the one or more nucleic acids is increased as compared to the transfection of the cells performed under the same conditions but in the absence of the stabilizer.

In some aspects, a method for producing high titer recombinant adeno-associated viral (rAAV) vector is provided. The method comprises the following steps: (i) preparing a transfection cocktail comprising one or more cationic polymers, a stabilizer and one or more nucleic acids, (ii) contacting the transfection cocktail prepared in step (i) with cells to be transfected to form a mixture, (iii) incubating the mixture of step (ii) to make transfected cells that produce rAAV vector, and (iv) isolating and/or purifying rAAV vector from the transfected cells produced in step (iii), wherein the time between initiation of step (i) and completion of step (ii) is greater than 10 seconds, and wherein the rAAV titer is increased by at least 2-fold and/or by at least 5% as compared to the rAAV titer produced under the same conditions but in the absence of the stabilizer.

The time between initiation of step (i) (i.e., preparation of the transfection cocktail) and completion of step (ii) (i.e., contacting the transfection cocktail prepared in step (i) with cells to be transfected to form a mixture) can be anywhere between about 10 seconds to about 10 days. In some embodiments, the time between initiation of step (i) and completion of step (ii) is between about 15 seconds to about 5 days, about 30 seconds to about 2 days, about 60 seconds to about 1 day, about 90 seconds to about 10 hours, about 90 seconds to about 8 hours, about 2 minutes to about 4 hours, about 4 minutes to about 2 hours, about 6 minutes to about 1 hour, or about 10 minutes to about 30 minutes. In some embodiments, the time between initiation of step (i) and completion of step (ii) is more than about 1 minute, more than about 2 minutes, more than about 6 minutes, more than about 8 minutes, more than about 10 minutes, more than 15 minutes, more than about 30 minutes, more than about 1 hour, more than about 2 hours, more than about 6 hours, more than about 8 hours, more than about 10 hours, more than about 12 hours, more than about 24 hours, or more than 2 days.

In some embodiments, the time between initiation of step (i) and completion of step (ii) is 2 minutes. In some embodiments, the time between initiation of step (i) and completion of step (ii) is 10 minutes. In some embodiments, the time between initiation of step (i) and completion of step (ii) is 30 minutes. In some embodiments, the time between initiation of step (i) and completion of step (ii) is more than 2 minutes, more than 10 minutes or more than 30 minutes.

In some embodiments, the time between initiation of step (i) and completion of step (ii) does not include a transfection cocktail incubation time. In some embodiments, the time between initiation of step (i) and completion of step (ii) includes a transfection cocktail incubation time. As used herein, the term “transfection cocktail incubation time” refers to the time the prepared transfection cocktail of step (i) is incubated before contacting with the cells to be transfected. In some embodiments, the transfection cocktail of step (i) is incubated from about 10 seconds to about 10 days, about 15 seconds to about 5 days, about 30 seconds to about 2 days, about 60 seconds to about 1 day, about 90 seconds to about 10 hours, about 90 seconds to about 8 hours, about 2 minutes to about 4 hours, about 4 minutes to about 2 hours, about 6 minutes to about 1 hour, or about 10 minutes to about 30 minutes prior to step (ii). In some embodiments, the transfection cocktail of step (i) is incubated for more than about 1 minute, more than about 2 minutes, more than about 6 minutes, more than about 8 minutes, more than about 10 minutes, more than 15 minutes, more than about 30 minutes, more than about 1 hour, more than about 2 hours, more than about 6 hours, more than about 8 hours, more than about 10 hours, more than about 12 hours, more than about 24 hours, or more than 2 days prior to step (ii).

In some embodiments, the transfection cocktail is incubated at about 4° C. to about room temperature prior to step (ii). In some embodiments, the transfection cocktail is incubated at about room temperature prior to step (ii).

The transfection cocktail comprises one or more cationic polymers, a stabilizer and one or more nucleic acids. In some embodiments, the transfection cocktail is prepared by first mixing the one or more cationic polymers with the stabilizer to form a resultant mixture which is then added to the one or more nucleic acids. In some embodiments, the transfection cocktail is prepared by first mixing the stabilizer with the one or more nucleic acids to form a resultant mixture which is then added to the one or more cationic polymers. In some embodiments, the transfection cocktail is prepared by first mixing the one or more cationic polymers with the one or more nucleic acids to form a resultant mixture which is then added to the stabilizer. For cells transfected at a high cell density (e.g., more than about 18×10⁶ cells/mL), the transfection cocktail may be prepared by first mixing the stabilizer with the one or more nucleic acids to form a resultant mixture which is then added to the one or more cationic polymers. For cells transfected at a low cell density (e.g., less than about 18×10⁶ cells/mL), the transfection cocktail can be prepared by mixing its components (i.e., the nucleic acids, the cationic polymer and the stabilizer) in any order.

In some embodiments, the resultant mixture is added to the one or more nucleic acids, the one or more cationic polymers or the stabilizer with no mixing. In some embodiments, the resultant mixture is added to the one or more nucleic acids, the one or more cationic polymers or the stabilizer and mixed at about 10 revolutions per minute (rpm), about 20 rpm, about 25 rpm, about 30 rpm, about 35 rpm, about 40 rpm, about 45 rpm, about 50 rpm, about 55 rpm, about 60 rpm, about 65 rpm, about 70 rpm, about 75 rpm, about 80 rpm, about 85 rpm, about 90 rpm, about 95 rpm, about 100 rpm, about 110 rpm, about 120 rpm, about 130 rpm, about 140 rpm, about 150 rpm, about 200 rpm, about 300 rpm, about 400 rpm or about 500 rpm. In some embodiments, the the resultant mixture is added to the one or more nucleic acids, the one or more cationic polymers or the stabilizer and mixed at about 120 rpm.

The transfection cocktail may be prepared at from about 4° C. to about room temperature. In some embodiments, the transfection cocktail is prepared at about room temperature. The term ‘room temperature’ as used herein refers to temperatures in the range of 20° C. to 25° C.

As used herein, the term “cationic polymer” is intended to refer to positively charged polymers having the capacity to condense nucleic acid (e.g. DNA). Cationic polymers include polyelectrolytes and cationic polypeptides. Nonlimiting examples of cationic polymers include chitosan, poly-L-lysine (pLL), polyamine (PA), polyalkylenimine (PAI), polyethylenimine (PEI), poly[a-(-aminobutyl)-L-glycolic acid], polyamidoamine, poly(2-dimethylamino)ethyl methacrylate, polyhistidine, polyarginine, poly(4-vinylpyridine), poly(vinylamine) and poly(4-vinyl-N-alkyl pyridinium halide).

The cationic polymer may be branched or linear. In some embodiments, the cationic polymer has a molecular weight ranging from about 500 Da to about 160,000 Da and/or about 2,500 Da to about 250,000 Da in free base form.

In some embodiments, the cationic polymer is polyethylenimine (PEI) or poly-L-lysine (pLL). In some embodiments, the cationic polymer is polyethylenimine (PEI). PEI can be linear PEI or branched PEI. PEI can be in a salt form or free base. In some embodiments, PEI is linear PEI, such as an optionally hydrolyzed linear PEI. The hydrolyzed PEI may be fully or partially hydrolyzed. Hydrolyzed linear PEI has a greater proportion of free (protonatable) nitrogens compared to non-hydrolyzed linear PEI, typically having at least 1-5% more free (protonatable) nitrogens compared to non-hydrolyzed linear PEI, more typically having 5-10% more free (protonatable) nitrogens compared to non-hydrolyzed linear PEI, or most typically having 10-15% more free (protonatable) nitrogens compared to non-hydrolyzed linear PEI.

In some embodiments, PEI can have a molecular weight in the range of about 4,000 to about 160,000 and/or in the range of about 2,500 to about 250,000 molecular weight in free base form. In some embodiments, PEI can have a molecular weight of about 40,000 and/or about 25,000 molecular weight in free base form. In some embodiments, PEI can have a molecular weight of about 40,000 and/or about 22,000 molecular weight in free base form. In some embodiments, the cationic polymer comprises hydrolyzed linear PEI with a molecular weight of about 40,000 and/or about 22,000 molecular weight in free base form. In addition, chemically modified linear PEI or branched PEI can be also used. In some embodiments, the PEI has a fully depropionylated structure. PEI is commercially available (e.g., Polysciences, Inc., Warrington, Pa., USA). In some embodiments, the PEI is PEImax.

As used herein, the term “stabilizer” comprises a cationic polymer grafted (i.e., conjugated) with a neutral moiety. In some embodiments, the neutral moiety comprises poly(ethylene glycol) (PEG) or albumin. In some embodiments, the neutral moiety comprises PEG. The cationic polymer grafted with the neutral moiety may be selected from any of the cationic polymers described herein.

In some embodiments, the stabilizer comprises a cationic polymer grafted with a poly(ethylene glycol) (PEG) moiety. In some embodiments, PEG has a molecular weight from about 250 Da to 35,000 Da. In some embodiments, the cationic polymer is PEI or pLL. In some embodiments, the cationic polymer grafted with PEG is not PEI. In some embodiments, the cationic polymer grafted with PEG is not pLL.

In some embodiments, the stabilizer comprises branched PEI with a molecular weight from about 2,000 to about 60,000 grafted with PEG having a molecular weight from about 250 Da to 35,000 Da. In some embodiments, the stabilizer comprises branched PEI with a molecular weight of about 20,000 to about 25,000 Da grafted with PEG having a molecular weight from about 250 Da to about 5,000 Da. In some embodiments, the stabilizer comprises branched PEI with a molecular weight of about 25,000 Da grafted with PEG having a molecular weight of about 5,000 Da. In some embodiments, the stabilizer comprises branched PEI with a molecular weight of about 25,000 Da grafted with PEG having a molecular weight of about 500 Da.

In some embodiments, the stabilizer comprises linear PEI with a molecular weight from about 2,000 to about 60,000 grafted with PEG having a molecular weight from about 250 Da to 35,000 Da. In some embodiments, the stabilizer comprises linear PEI with a molecular weight of about 20,000 Da grafted with PEG having a molecular weight of about 5,000 Da. In some embodiments, the stabilizer comprises linear PEI with a molecular weight of about 20,000 Da grafted with PEG having a molecular weight of about 500 Da. In some embodiments, the stabilizer comprises pLL with a molecular weight of about 26000 Da conjugated to PEG having a molecular weight of about 5000 Da.

In some embodiments, the number of PEG moieties grafted per molecule of cationic polymer is 1, 5, 10, 15, 20, 25, 30, 35, 40, 45, 50, 55, 60, 65, 70, 75, 80, 85, 90, 95, 100 or more. In some embodiments, the number of PEG moieties grafted per molecule of cationic polymer is 1, 5, 15 or 55.

When contacted with the transfection cocktail in step (ii) of any of the methods described herein, the cells may be at a cell density of at least 1×10⁵ cells/mL, at least 2×10⁵ cells/mL, at least 4×10⁵ cells/mL, at least 6×10⁵ cells/mL, at least 8×10⁵ cells/mL, at least 0.5×10⁶ cells/mL, at least 1×10⁶ cells/mL, at least 2×10⁶ cells/mL, at least 4×10⁶ cells/mL, at least 6×10⁶ cells/mL, at least 8×10⁶ cells/mL, at least 10×10⁶ cells/mL, at least 12×10⁶ cells/mL, at least 14×10⁶ cells/mL, at least 16×10⁶ cells/mL, at least 18×10⁶ cells/mL, at least 20×10⁶ cells/mL, at least 22×10⁶ cells/mL, at least 24×10⁶ cells/mL, at least 26×10⁶ cells/mL, at least 28×10⁶ cells/mL, at least 30×10⁶ cells/mL, at least 32×10⁶ cells/mL, at least 34×10⁶ cells/mL, at least 36×10⁶ cells/mL, at least 38×10⁶ cells/mL, at least 40×10⁶ cells/mL, at least 42×10⁶ cells/mL, at least 44×10⁶ cells/mL, at least 46×10⁶ cells/mL, at least 48×10⁶ cells/mL, at least 50×10⁶ cells/mL, or at least 52×10⁶ cells/mL.

In some embodiments, the cells are at a density of at least 12×10⁶ cells/mL, at least 14×10⁶ cells/mL, at least 16×10⁶ cells/mL, at least 18×10⁶ cells/mL, at least 20×10⁶ cells/mL, at least 22×10⁶ cells/mL, at least 24×10⁶ cells/mL, at least 26×10⁶ cells/mL, at least 28×10⁶ cells/mL, or at least 30×10⁶ cells/mL, at least 32×10⁶ cells/mL, at least 34×10⁶ cells/mL, at least 36×10⁶ cells/mL, at least 38×10⁶ cells/mL, at least 40×10⁶ cells/mL, at least 42×10⁶ cells/mL, at least 44×10⁶ cells/mL, at least 46×10⁶ cells/mL, or at least 48×10⁶ cells/mL, when contacted with the transfection cocktail in step (ii).

In some embodiments, the cells are at a density of at least 24×10⁶ cells/mL, at least 26×10⁶ cells/mL, at least 28×10⁶ cells/mL, at least 30×10⁶ cells/mL, at least 32×10⁶ cells/mL, at least 34×10⁶ cells/mL, at least 36×10⁶ cells/mL, at least 38×10⁶ cells/mL, at least 40×10⁶ cells/mL, at least 42×10⁶ cells/mL, at least 44×10⁶ cells/mL, at least 46×10⁶ cells/mL, or at least 48×10⁶ cells/mL when contacted with the transfection cocktail in step (ii).

In some embodiments, the one or more cationic polymers are used in an amount of about 0.05 μg per million cells, about 0.1 μg per million cells, about 0.2 μg per million cells, about 0.4 μg per million cells, about 0.6 μg per million cells, about 0.8 μg per million cells, about 1.0 μg per million cells, about 1.5 μg per million cells, about 2 μg per million cells, about 2.1 μg per million cells, about 2.2 μg per million cells, about 2.3 μg per million cells, about 2.4 μg per million cells, about 2.5 μg per million cells, about 2.6 μg per million cells, about 2.7 μg per million cells, about 2.8 μg per million cells, about 2.9 μg per million cells, about 3.0 μg per million cells, about 3.5 μg per million cells, about 4.0 μg per million cells, about 4.5 μg per million cells, about 5 μg per million cells, about 5.5 μg per million cells, about 6.0 μg per million cells, about 6.5 μg per million cells, about 7.0 μg per million cells, about 7.5 μg per million cells, about 8.0 μg per million cells, about 10 μg per million cells.

In some embodiments, the one or more cationic polymers are used in an amount of about 2.2 μg per million cells.

In some embodiments, the one or more cationic polymers are used in an amount of about about 0.01 μg, about 0.02 μg, about 0.04 μg, about 0.06 μg, about 0.08 μg, about 1.0 μg, about 1.2 μg, about 1.4 μg, about 1.6 μg, about 1.8 μg, about 2.0 μg, about 2.2 μg, about 2.4 μg, about 2.6 μg, about 2.8 μg, about 3.0 μg, about 3.2 μg, about 3.4 μg, about 3.6 μg, about 3.8 μg, about 4.0 μg, about 4.2 μg, about 4.6 μg, about 4.8 μg, about 5.0 μg, about 5.5 μg, about 6.0 μg, about 6.5 μg, about 7 μg, about 7.5 μg, about 8.0 μg, about 8.5 μg, about 9.0 μg, about 9.5 μg, about 10 μg, about 12 μg, about 14 μg, about 16 μg, about 18 μg, about 20 μg, about 40 μg, about 60 μg, about 80 μg, about 100 μg, about 0.1 mg, about 1 mg, about 2 mg, about 4 mg, about 6 mg, about 8 mg or about 10 mg. In some embodiments, the about 20 μg, about 22 μg, about 24 μg, about 26 μg, about 26.4 μg, about 28 μg, about 30 μg, about 32 μg, about 34 μg, about 36 μg, about 38 μg, about 40 μg, about 42 μg, about 44 μg, about 46 μg, about 48 μg, about 50 μg, about 52 μg, about 52.8 μg, about 54 μg, about 56 μg, about 58 μg, or about 60 μg.

In some embodiments, the amount of stabilizer in the transfection cocktail is about 1%, about 2%, about 2.5%, about 5%, about 7.5%, about 10%, about 12.5%, about 15%, about 17.5%, about 20%, about 25%, about 30%, about 40%, about 50%, about 60%, about 70%, about 75%, about 80%, about 90%, about 100%, about 200%, about 300%, about 400%, or about 500% relative to the amount of the one or more cationic polymers.

In some embodiments, the amount of stabilizer in the transfection cocktail is about 0.01 fold, about 0.05 fold, about 0.1 fold, about 0.2-fold, about 0.4 fold, about 0.6 fold, about 0.8 fold, about 1-fold, about 5-fold, about 10-fold, about 100 fold, about 200 fold, about 300 fold, about 400 fold, about 500 fold, or about 600 fold relative to the amount of the one or more cationic polymers.

In some embodiments, the amount of stabilizer in the transfection cocktail is about 5%, about 5.5%, about 6%, about 6.5%, about 7%, about 7.5%, about 8%, about 8.5%, about 9%, about 9.5%, about 10%, about 12.5%, about 15%, about 17.5%, about 20%, about 25%, about 30% or about 40% relative to the amount of the one or more cationic polymers.

In other embodiments, the amount of stabilizer in the transfection cocktail is between about 5% and 7.5%, 5% and 10%, 5% and 12.5%, or 5% and 15% relative to the amount of the one or more cationic polymers. In other embodiments, the amount of stabilizer in the transfection cocktail is between about 7.5% and 10%, 7.5% and 12.5%, or 7.5% and 15% relative to the amount of the one or more cationic polymers. In other embodiments, the amount of stabilizer in the transfection cocktail is between about 10% and 12.5%, or 10% and 15% relative to the amount of the one or more cationic polymers. In each of the foregoing embodiments, the cationic polymer may be PEI and the stabilizer may be a branched or linear PEI conjugated with PEG. In each of the foregoing embodiments, the cationic polymer is PEI and the stabilizer is branched PEI conjugated with PEG. In each of the foregoing embodiments, the cationic polymer is PEI and the stabilizer is branched PEI conjugated with PEG, wherein the average number of PEG moeities per PEI is fewer than about 55, 50, 45, 40, 35, 30, 25, 20, or 15. In each of the foregoing embodiments, the cationic polymer is PEI and the stabilizer is branched PEI conjugated with PEG, wherein the average number of PEG moeities per PEI is about 15. For each of the foregoing embodiments, the disclosure further provides combinations of transfection cocktail and the cells to be transfected therewith.

In other embodiments, the amount of stabilizer in the transfection cocktail is between about 10% and 25%, 10% and 20%, or 10% and 15% relative to the amount of the one or more cationic polymers. In other embodiments, the amount of stabilizer in the transfection cocktail is between about 15% and 25%, or 15% and 20% relative to the amount of the one or more cationic polymers. In each of the foregoing embodiments, the cationic polymer may be PEI and the stabilizer may be a branched or linear PEI conjugated with PEG. In each of the foregoing embodiments, the cationic polymer is PEI and the stabilizer is branched PEI conjugated with PEG. In each of the foregoing embodiments, the cationic polymer is PEI and the stabilizer is branched PEI conjugated with PEG, wherein the average number of PEG moeities per PEI is fewer than about 55, 50, 45, 40, 35, 30, 25, 20, or 15. In each of the foregoing embodiments, the cationic polymer is PEI and the stabilizer is branched PEI conjugated with PEG, wherein the average number of PEG moeities per PEI is about 15. For each of the foregoing embodiments, the disclosure further provides combinations of transfection cocktail and the cells to be transfected therewith.

In some embodiments, the amount of stabilizer in the transfection cocktail is about 0.01 μg, about 0.02 μg, about 0.04 μg, about 0.06 μg, about 0.08 μg, about 1.0 μg, about 1.2 μg, about 1.4 μg, about 1.6 μg, about 1.8 μg, about 2.0 μg, about 2.2 μg, about 2.4 μg, about 2.6 μg, about 2.8 μg, about 3.0 μg, about 3.2 μg, about 3.4 μg, about 3.6 μg, about 3.8 μg, about 4.0 μg, about 4.2 μg, about 4.6 μg, about 4.8 μg, about 5.0 μg, about 5.5 μg, about 6.0 μg, about 6.5 μg, about 7 μg, about 7.5 μg, about 8.0 μg, about 8.5 μg, about 9.0 μg, about 9.5 μg, about 10 μg, about 12 μg, about 14 μg, about 16 μg, about 18 μg, about 20 μg, about 40 μg, about 60 μg, about 80 μg, about 100 μg, about 0.1 mg, about 1 mg, about 2 mg, about 4 mg, about 6 mg, about 8 mg or about 10 mg.

The amount of stabilizer used may depend on the cell density at transfection (i.e., when contacted with the transfection cocktail in step (ii) of any of the methods described herein). In some embodiments, when the cells are transfected at low cell density (for example, but not limited to, less than about 18×10⁶ cells/mL), the amount of stabilizer in the transfection cocktail is about 7.5% to about 15% relative to the amount of the one or more cationic polymers used in the method. In some embodiments, when the cells are transfected at low cell density, the amount of stabilizer in the transfection cocktail is about 7.5%, about 10%, about 12.5%, or about 15%, relative to the amount of the one or more cationic polymers used in the method. In some embodiments, when the cells are transfected at low cell density, the amount of stabilizer in the transfection cocktail is about 7.5% to about 10%, relative to the amount of the one or more cationic polymers used in the method. In some embodiments, when the cells are transfected at low cell density, the amount of stabilizer in the transfection cocktail is 10%, relative to the amount of the one or more cationic polymers used in the method.

In some embodiments, the cells are at a density of about 12×10⁶ cells/mL when contacted with the transfection cocktail in step (ii) and the amount of stabilizer in the transfection cocktail is about 10% relative to the amount of the one or more cationic polymers. In some embodiments, the stabilizer comprises branched PEI with a molecular weight of about 25,000 Da grafted with PEG having a molecular weight of about 5,000 Da.

In some embodiments, when the cells are transfected at high cell density (for example, but not limited to, more than about 18×10⁶ cells/mL), the amount of stabilizer in the transfection cocktail is about 15% to about 30% relative to the amount of the one or more cationic polymers used in the method. In some embodiments, when the cells are transfected at high cell density, the amount of stabilizer in the transfection cocktail is about 15%, about 20%, about 25%, or about 30%, relative to the amount of the one or more cationic polymers used in the method. In some embodiments, when the cells are transfected at high cell density, the amount of stabilizer in the transfection cocktail is about 25%, relative to the amount of the one or more cationic polymers used in the method.

In some embodiments, the cells are at a density of at least 24×10⁶ cells/mL when contacted with the transfection cocktail in step (ii) and the amount of stabilizer in the transfection cocktail is about 25% relative to the amount of the one or more cationic polymers. In some embodiments, the stabilizer comprises branched PEI with a molecular weight of about 25,000 Da grafted with PEG having a molecular weight of about 5,000 Da.

In other embodiments, cells at high density (thus, at least about 18×10⁶ cells/mL, 19×10⁶ cells/mL, 20×10⁶ cells/mL, 21×10⁶ cells/mL, 22×10⁶ cells/mL, 23×10⁶ cells/mL, 24×10⁶ cells/mL, 25×10⁶ cells/mL, 26×10⁶ cells/mL, 27×10⁶ cells/mL, 28×10⁶ cells/mL, 29×10⁶ cells/mL, 30×10⁶ cells/mL, or more) can be transfected using a transfection cocktail comprising nucleic acid(s) cationic polymer and stabilizer, where the amount of stabilizer in the transfection cocktail is between about 10% and 25%, 10% and 20%, 10% and 15%, 15% and 25%, or 15% and 20% relative to the amount of cationic polymer. In each of the foregoing embodiments, the cationic polymer may be PEI and the stabilizer may be a branched or linear PEI conjugated with PEG. In each of the foregoing embodiments, the cationic polymer is PEI and the stabilizer is branched PEI conjugated with PEG. In each of the foregoing embodiments, the cationic polymer is PEI and the stabilizer is branched PEI conjugated with PEG, wherein the average number of PEG moeities per PEI is fewer than about 55, 50, 45, 40, 35, 30, 25, 20, or 15. In each of the foregoing embodiments, the cationic polymer is PEI and the stabilizer is branched PEI conjugated with PEG, wherein the average number of PEG moeities per PEI is about 15. In each of the foregoing embodiments, the transfection cocktail is incubated for at least 7 minutes, at least 8 minutes, at least 9 minutes, at least 10 minutes, or longer, before contacting the cells. In each of the foregoing embodiments, the transfection cocktail can be prepared as a combination of submixtures, such that a Mix A, comprising nucleic acids (such as plasmids) and cell growth media is prepared, and separately a Mix B, comprising cationic polymer (such as PEI) and cell growth media is prepared, after which stabilizer is added to and mixed with Mix A first, and only thereafter is Mix B added and mixed with the earlier combination of Mix A and stabilizer. Then, the combination of Mix A, stabilizer and Mix B is contacted to cells for transfection.

According to certain other embodiments, the disclosure provides methods of cellular transfection comprising contacting cells at a density of at least about 18×10⁶ cells/mL, or between about 20×10⁶ cells/mL and 28×10⁶ cells/mL, or between about 22×10⁶ cells/mL and 26×10⁶ cells/mL, or about 24×10⁶ cells/mL, with a transfection cocktail comprising plasmid DNA, PEI and a stabilizer, where the amount of stabilizer in the transfection cocktail is at least 10%, or between about 20% and 30%, or between about 22.5% and 27.5%, or about 25% relative to the amount of PEI, and where the stabilizer is branched PEI conjugated with PEG, where the average number of PEG moeities per PEI is fewer than about 30, 25, 20, or 15, or where the average number of PEG moeities per PEI is about 15. In each of the foregoing embodiments, the transfection cocktail is incubated for at least 7 minutes before being contacted with the cells. Furthermore, in each of the foregoing embodiments, the transfection cocktail can be prepared as a combination of submixtures, such that a Mix A, comprising the plasmids and cell growth media is prepared, and separately a Mix B, comprising the PEI and cell growth media is prepared, after which the stabilizer is added to and mixed with Mix A first, and only thereafter is Mix B added and mixed with the earlier combination of Mix A and stabilizer. Then, the combination of Mix A, stabilizer and Mix B is contacted to cells for transfection (either in one bolus, or continuously until the volume of transfection cocktail is exhausted). In each of the foregoing embodiments, the transfection cocktail may, in certain additional embodiments, comprise one or more plasmids comprising the genetic sequences required for recombinant AAV vector production, including without limitation rep, cap, adenoviral or other viral helper functions, and a vector genome including inverted terminal repeats. Related to the foregoing methods, the disclosure further provides combinations of transfection cocktail and the cells to be transfected therewith. In other embodiments, the transfection cocktail comprises a plasmid or other nucleic acid configured as an expression construct for expression of a recombinant therapeutic protein, including but not limited to an antibody or antigen binding fragment thereof.

In some embodiments, the amount of stabilizer in the transfection cocktail is insufficient to transfect the cells when used alone (i.e., without one or more cationic polymers). For example, the transfection efficiency obtained with stabilizer alone is lower (e.g., by at least 1.5-fold, by at least 2-fold, by at least 4-fold, by at least 6-fold, by at least 8-fold or by at least 10-fold) as compared to that obtained with a cationic polymer (e.g., PEI) alone.

The one or more nucleic acids may be used in an amount of about 0.05 μg per million cells, about 0.1 μg per million cells, about 0.2 μg per million cells, about 0.4 μg per million cells, about 0.5 μg per million cells, about 0.6 μg per million cells, about 0.8 μg per million cells, about 1.0 μg per million cells, about 1.5 μg per million cells, about 2.0 μg per million cells, about 2.5 μg per million cells, about 3.0 μg per million cells, about 3.5 μg per million cells, about 4.0 μg per million cells, about 4.5 μg per million cells, about 5 μg per million cells, about 5.5 μg per million cells, about 6.0 μg per million cells, about 6.5 μg per million cells, about 7.0 μg per million cells, about 7.5 μg per million cells, about 8.0 μg per million cells, about 8.5 μg per million cells, about 9.0 μg per million cells, about 9.5 μg per million cells, or about 10 μg per million cells in any of the methods described herein. In some embodiments, the one or more nucleic acids are used in an amount of about 1.0 μg per million cells. In some embodiments, the one or more nucleic acids are used in an amount of about 12 μg or about 24 μg.

The one or more cationic polymers and the one or more nucleic acids are mixed in a ratio that is not limited. For example, the one or more nucleic acids and the one or more cationic polymers are used in a weight (or molar) ratio in the range of about 1:0.01 to about 1:100, or in a weight (or molar) ratio of about 0.01:1 to about 100:1. In some embodiments, the one or more cationic polymers and the one or more nucleic acids are present in a weight (or molar) ratio in the range of about 1:1 to 50:1. In some embodiments, the one or more nucleic acids and the one or more cationic polymers are present in a weight (or molar) ratio in the range of about 1:1 to 1:5.

In some embodiments, the one or more nucleic acids and the one or more cationic polymers are present in a weight (or molar) ratio of about 1:2.2.

In the methods of the invention, the transfection cocktail is contacted with cells to be transfected to form a mixture and the mixture is incubated to achieve cell transfection. The incubation time after cell are contacted with the transfection cocktail can range from seconds to days. In some embodiments, the mixture is incubated for about 2 minutes to about 150 hours to make transfected cells. In some embodiments, the mixture is incubated for about 15 minutes to about 150 hours to make transfected cells.

In some embodiments, the mixture in step (iii) of the methods of the invention is incubated for about 15 minutes, 30 minutes, 1 hour, 2 hours, 3 hours, 4 hours, 5 hours, 6 hours, 7 hours, 8 hours, 9 hours, 10 hours, 20 hours, 40 hours, 60 hours, 80 hours, 100 hours, 120 hours, 140 hours or 150 hours to make transfected cells. In some embodiments, the mixture in step (iii) is incubated for 1 hour, 2 hours, 3 hours, 4 hours, or 5 hours to make transfected cells.

The longer the incubation time of the cells with the transfection cocktail, the higher the likelihood that the cells may be affected by cytotoxicity of the cationic polymer (such as PEI) resulting in an increased amount of dead (non-viable) cells thereby reducing transfection efficiency.

To reduce cytotoxicity of PEI, culture medium may be replaced with fresh culture medium after contacting the cells with the transfection cocktail. Culture medium replacement after transfection can minimize PEI cytotoxicity without significant loss of cell transfection efficiency.

Numerous cell growth medium appropriate for sustaining cell viability or providing cell growth and/or proliferation are commercially available or can be readily produced. Examples of such medium include serum free eukaryotic growth mediums, such as medium for sustaining viability or providing for the growth of mammalian (e.g., human) cells. Non-limiting examples include Ham's F12 or F12K medium (Sigma-Aldrich), FreeStyle™ (FS) F17 medium (Thermo-Fisher Scientific), MEM, DMEM, RPMI-1640 (Thermo-Fisher Scientific) and mixtures thereof. Such medium can be supplemented with vitamins and/or trace minerals and/or salts and/or amino acids, such as essential amino acids for mammalian (e.g., human) cells.

Cells to be transfected (i.e., “host cells”) by the methods and compositions of the invention include, but are not limited to, microorganisms, yeast cells, insect cells, and mammalian cells (e.g., human cells). Such cells may be primary cells or cell lines that are capable of growth or maintaining viability in vitro, or have been adapted for in vitro tissue culture. Examples of cell lines include HEK (human embryonic kidney) cells, Chinese hamster ovary (CHO) cells, or insect-derived Sf9 cells. In some embodiments, the cells comprise Human Embryonic Kidney (HEK) 293 cells. In some embodiments, the cells comprise HEK 293E, HEK 293F or HEK 293T cells.

The cells (e.g., HEK 293 cells) may be in suspension culture or may be adherent. The cells may be adapted for serum-free growth in suspension. In some embodiments, the cells are grown or maintained in a serum-free culture medium. In some embodiments, the cells are stably or transiently transfected.

The cells may be grown or maintained in roller bottles or expanded roller bottles. In some embodiments, the cells are grown in bioreactors. In some embodiments, the cells are grown in bags or flasks. In some embodiments, the cells are grown in a WAVE bioreactor. In some embodiments, the cells are grown in a stirred tank bioreactor.

The one or more nucleic acid molecules used in the methods of the invention may comprise one or more plasmids. In some embodiments, the one or more nucleic acids or the one or more plasmids encode a therapeutic moiety. In some embodiments, the therapeutic moiety comprises a protein or a transcript. Non-limiting examples of therapeutic proteins are described herein and include antibodies, cytokines, receptors, growth factors, clotting factors, transporters etc. Non-limiting examples of transcripts are described herein and include inhibitory RNA (RNAi, e.g., small or short hairpin (sh)RNA, microRNA (miRNA), small or short interfering (si)RNA, trans-splicing RNA, or antisense RNA).

In some embodiments, the one or more nucleic acid molecules comprise (a) one or more plasmids comprising nucleic acids encoding AAV packaging proteins and/or nucleic acids encoding helper proteins, and/or (b) a plasmid comprising a nucleic acid encoding a transgene of interest. In some embodiments, the one or more plasmids of (a) comprise a first plasmid comprising the nucleic acids encoding AAV packaging proteins and a second plasmid comprising the nucleic acids encoding helper proteins.

The molar ratio of the plasmid comprising the transgene to the first plasmid comprising the nucleic acids encoding AAV packaging proteins to the second plasmid comprising the nucleic acids encoding helper proteins may be in the range of about 1-10:1:1, or 1:1-10:1, or 1:1:1-10.

In some embodiments, the encoded AAV packaging proteins comprise AAV rep and/or AAV cap proteins.

In some embodiments, the encoded helper proteins comprise adenovirus E2 and/or E4, VARNA proteins, and/or non-AAV helper proteins.

The transgene includes any nucleic acid, such as a gene that is transcribed into a transcript or that encodes a polypeptide or protein of interest. For example, but not limited to, the transgene encodes a wild type or functional variant blood clotting factor, mini-dystrophin, C1 esterase inhibitor, copper transporting P-type ATPase (ATP7B), or copper-zinc superoxide dismutase 1 (SOD1). In some embodiments, the wild type or functional variant blood clotting factor is Factor VII, Factor VIII or Factor IX.

The methods described herein may further comprise a step of culturing, expanding, isolating or selecting for cells that have been transfected with the one or more nucleic acids. The transfected cells so produced and/or the culture medium from the transfected cells so produced may be harvested.

The methods described herein may also further comprise a step of isolating and/or purifying rAAV vector from the transfected cells so produced. Following cell transfection and/or production of recombinant viral (e. AAV) vectors as set forth herein, if desired the viral (e.g., rAAV) virions can be collected/harvested from the cells/cell culture and optionally purified and/or isolated from transfected cells using a variety of conventional methods. Such methods include column chromatography, CsCI gradients, and the like. For example, a plurality of column purification steps such as purification over an anion exchange column, an affinity column and/or a cation exchange column can be used. (See, e.g., International Publication No. WO 02/12455 and US Application Publication Nos. 20030207439). Alternatively, or in addition, CsCI gradient steps can be used. (See, e.g., US Application Publication Nos. 20120135515; and 20130072548) Further, if the use of infectious virus is employed to express the packaging and/or helper proteins, residual virus can be inactivated, using various methods. For example, adenovirus can be inactivated by heating, to temperatures of approximately 60° C. for, e.g., 20 minutes or more. This treatment effectively inactivates the helper virus since AAV is heat stable while the helper adenovirus is heat labile.

The methods described herein may increase transfection of the cells with the one or more nucleic acids by at least 5%, at least 10%, at least 15%, at least 20%, at least 25%, at least 30%, at least 35%, at least 35%, at least 40%, at least 45%, or at least 50% as compared to the transfection of the cells performed under the same conditions but in the absence of the stabilizer.

The methods described herein may increase the amount of rAAV vector isolated/purified from the transfected cells by at least 5%, at least 10%, at least 15%, at least 20%, at least 25%, at least 30%, at least 35%, at least 35%, at least 40%, at least 45%, or at least 50% or greater as compared to the amount of rAAV vector isolated/purified from the transfected cells under the same conditions but in the absence of the stabilizer.

The methods described herein may increase the amount of rAAV vector isolated/purified from the transfected cells by at least 2-fold, at least 4-fold, at least 10-fold, at least 20-fold, at least 40-fold, at least 50-fold, at least 60-fold, at least 80-fold, at least 100-fold or greater as compared to the amount of rAAV vector isolated/purified from the transfected cells under the same conditions but in the absence of the stabilizer.

The methods described herein may increase the rAAV titer by at least 2-fold at least 4-fold, at least 10-fold, at least 20-fold, at least 40-fold, at least 50-fold, at least 60-fold, at least 80-fold, at least 100-fold or greater as compared to the rAAV titer produced under the same conditions but in the absence of the stabilizer.

The methods described herein may increase the rAAV titer by at least 5%, at least 10%, at least 15%, at least 20%, at least 25%, at least 30%, at least 35%, at least 35%, at least 40%, at least 45%, or at least 50% or greater as compared to the rAAV titer produced under the same conditions but in the absence of the stabilizer.

The methods described herein may increase the number of rAAV full vectors by at least 5%, at least 10%, at least 15%, at least 20%, at least 25%, at least 30%, at least 35%, at least 35%, at least 40%, at least 45%, or at least 50% or greater as compared to the number of rAAV full vectors produced under the same conditions but in the absence of the stabilizer.

The methods described herein may decrease the number of rAAV empty by at least 5%, at least 10%, at least 15%, at least 20%, at least 25%, at least 30%, at least 35%, at least 35%, at least 40%, at least 45%, or at least 50% or greater as compared to the number of rAAV empty vectors produced under the same conditions but in the absence of the stabilizer.

The number of full/empty ratios may be determined by measuring the 260:280 ratio. For example, the rAAV vectors may be purified using size exclusion high pressure liquid chromatography (SE-HPLC) and the elution peaks compared between 260 nm and 280 nm to determine the ratio of empty to full vectors (See, for example, Sommer et al. (2003) Quantification of adeno-associated virus particles and empty capsids by optical density measurement. Molecular Therapy, Vol 7, Issue 1, P122-128). A comparison of the 260:280 ratio of empty, intermediate and full vectors revealed that the empty vectors have a 260:280 ratio close to 0.6, the intermediates have a 260:280 ratio between 0.9 and 1.1, while the full vectors have a 260:280 ratio close to 1.3 to 1.4.

In some embodiments, the rAAV vectors isolated and/or purified in the presence of the stabilizer have a 260:280 ratio of about 1.0 to about 1.2, while the rAAV vectors isolated and/or purified under the same conditions but in the absence of the stabilizer have a 260:280 ratio of about 0.6.

Some aspects of the present disclosure also include compositions. In some embodiments, a composition comprising one or more cationic polymers and a stabilizer is provided. In some embodiments, the composition further comprises one or more nucleic acids and/or cells (e.g., cell that are to be transfected).

In some embodiments, a composition comprising a stabilizer and one or more nucleic acids is provided. In some embodiments, the composition further comprises one or more cationic polymers and/or cells (e.g., cells that are to be transfected).

In some embodiments, a composition comprising one or more cationic polymers, a stabilizer, one or more nucleic acids, and cells (e.g., cells that are to be transfected) is provided.

In some embodiments, the amount of one or more cationic polymers in the compositions described herein is about 0.01 μg, about 0.02 μg, about 0.04 μg, about 0.06 μg, about 0.08 μg, about 1.0 μg, about 1.2 μg, about 1.4 μg, about 1.6 μg, about 1.8 μg, about 2.0 μg, about 2.2 μg, about 2.4 μg, about 2.6 μg, about 2.8 μg, about 3.0 μg, about 3.2 μg, about 3.4 μg, about 3.6 μg, about 3.8 μg, about 4.0 μg, about 4.2 μg, about 4.6 μg, about 4.8 μg, about 5.0 μg, about 5.5 μg, about 6.0 μg, about 6.5 μg, about 7 μg, about 7.5 μg, about 8.0 μg, about 8.5 μg, about 9.0 μg, about 9.5 μg, about 10 μg, about 12 μg, about 14 μg, about 16 μg, about 18 μg, about 20 μg, about 40 μg, about 60 μg, about 80 μg, about 100 μg, about 0.1 mg, about 1 mg, about 2 mg, about 4 mg, about 6 mg, about 8 mg or about 10 mg. In some embodiments, the amount of one or more cationic polymers in the compositions described herein about 20 μg, about 22 μg, about 24 μg, about 26 μg, about 26.4 μg, about 28 μg, about 30 μg, about 32 μg, about 34 μg, about 36 μg, about 38 μg, about 40 μg, about 42 μg, about 44 μg, about 46 μg, about 48 μg, about 50 μg, about 52 μg, about 52.8 μg, about 54 μg, about 56 μg, about 58 μg, or about 60 μg.

In some embodiments, the amount of one or more cationic polymers in the compositions described herein is relative to the number of cells to transfected. For example, in some embodiments, the amount of one or more cationic polymers in the compositions described herein is about 0.05 μg per million cells, about 0.1 μg per million cells, about 0.2 μg per million cells, about 0.4 μg per million cells, about 0.6 μg per million cells, about 0.8 μg per million cells, about 1.0 μg per million cells, about 1.5 μg per million cells, about 2 μg per million cells, about 2.1 μg per million cells, about 2.2 μg per million cells, about 2.3 μg per million cells, about 2.4 μg per million cells, about 2.5 μg per million cells, about 2.6 μg per million cells, about 2.7 μg per million cells, about 2.8 μg per million cells, about 2.9 μg per million cells, about 3.0 μg per million cells, about 3.5 μg per million cells, about 4.0 μg per million cells, about 4.5 μg per million cells, about 5 μg per million cells, about 5.5 μg per million cells, about 6.0 μg per million cells, about 6.5 μg per million cells, about 7.0 μg per million cells, about 7.5 μg per million cells, about 8.0 μg per million cells, about 10 μg per million cells. In some embodiments, the amount of one or more cationic polymers in the compositions described herein is about 2.2 μg per million cells.

In some embodiments, the amount of stabilizer in the compositions described herein is about 0.01 μg, about 0.02 μg, about 0.04 μg, about 0.06 μg, about 0.08 μg, about 1.0 μg, about 1.2 μg, about 1.4 μg, about 1.6 μg, about 1.8 μg, about 2.0 μg, about 2.2 μg, about 2.4 μg, about 2.6 μg, about 2.8 μg, about 3.0 μg, about 3.2 μg, about 3.4 μg, about 3.6 μg, about 3.8 μg, about 4.0 μg, about 4.2 μg, about 4.6 μg, about 4.8 μg, about 5.0 μg, about 5.5 μg, about 6.0 μg, about 6.5 μg, about 7 μg, about 7.5 μg, about 8.0 μg, about 8.5 μg, about 9.0 μg, about 9.5 μg, about 10 μg, about 12 μg, about 14 μg, about 16 μg, about 18 μg, about 20 μg, about 40 μg, about 60 μg, about 80 μg, about 100 μg, about 0.1 mg, about 1 mg, about 2 mg, about 4 mg, about 6 mg, about 8 mg or about 10 mg.

In some embodiments, the amount of stabilizer in the compositions described herein is about 1.0 μg, about 1.2 μg, about 1.4 μg, about 1.6 μg, about 1.8 μg, about 2.0 μg, about 2.2 μg, about 2.4 μg, about 2.6 μg, about 2.64 μg, about 2.8 μg, about 3.0 μg, about 3.2 μg, about 3.4 μg, about 3.6 μg, about 3.8 μg, about 4.0 μg, about 4.2 μg, about 4.6 μg, about 4.8 μg, about 5.0 μg, about 5.5 μg, about 6.0 μg, about 6.5 μg, about 7 μg, about 7.5 μg, about 8.0 μg, about 8.5 μg, about 9.0 μg, about 9.5 μg, about 10 μg, about 12 μg, about 13 μg, about 14 μg, about 16 μg, about 18 μg or about 20 μg.

In some embodiments, the amount of stabilizer in the compositions described herein is relative to the amount of one or more cationic polymers. For example, in some embodiments, the amount of stabilizer in the compositions described herein is about 1%, about 2%, about 2.5%, about 5%, about 7.5%, about 10%, about 12.5%, about 15%, about 17.5%, about 20%, about 25%, about 30%, about 40%, about 50%, about 60%, about 70%, about 75%, about 80%, about 90%, about 100%, about 200%, about 300%, about 400%, or about 500% relative to the amount of the one or more cationic polymers. In some embodiments, the amount of stabilizer in the compositions described herein is about 7.5%, about 10%, about 12.5%, about 15%, about 17.5%, about 20%, about 25%, about 30% or about 40% relative to the amount of the one or more cationic polymers.

In some embodiments, the amount of one or more nucleic acids in the compositions described herein is about 0.01 μg, about 0.02 μg, about 0.04 μg, about 0.06 μg, about 0.08 μg, about 1.0 μg, about 1.2 μg, about 1.4 μg, about 1.6 μg, about 1.8 μg, about 2.0 μg, about 2.2 μg, about 2.4 μg, about 2.6 μg, about 2.8 μg, about 3.0 μg, about 3.2 μg, about 3.4 μg, about 3.6 μg, about 3.8 μg, about 4.0 μg, about 4.2 μg, about 4.6 μg, about 4.8 μg, about 5.0 μg, about 5.5 μg, about 6.0 μg, about 6.5 μg, about 7 μg, about 7.5 μg, about 8.0 μg, about 8.5 μg, about 9.0 μg, about 9.5 μg, about 10 μg, about 12 μg, about 14 μg, about 16 μg, about 18 μg, about 20 μg, about 40 μg, about 60 μg, about 80 μg, about 100 μg, about 0.1 mg, about 1 mg, about 2 mg, about 4 mg, about 6 mg, about 8 mg or about 10 mg. In some embodiments, the amount of one or more nucleic acids is about 12 μg or 24 μg.

In some embodiments, the amount of one or more nucleic acids in the compositions described herein is relative to the number of cells being transfected. For example, in some embodiments, the amount of one or more nucleic acids in the compositions described herein is about 0.05 μg per million cells, about 0.1 μg per million cells, about 0.2 μg per million cells, about 0.4 μg per million cells, about 0.5 μg per million cells, about 0.6 μg per million cells, about 0.8 μg per million cells, about 1.0 μg per million cells, about 1.5 μg per million cells, about 2.0 μg per million cells, about 2.5 μg per million cells, about 3.0 μg per million cells, about 3.5 μg per million cells, about 4.0 μg per million cells, about 4.5 μg per million cells, about 5 μg per million cells, about 5.5 μg per million cells, about 6.0 μg per million cells, about 6.5 μg per million cells, about 7.0 μg per million cells, about 7.5 μg per million cells, about 8.0 μg per million cells, about 8.5 μg per million cells, about 9.0 μg per million cells, about 9.5 μg per million cells, or about 10 μg per million cells. In some embodiments, the amount of one or more nucleic acids in the compositions described herein is about 1.0 μg per million cells.

In some embodiments, the cells in the compositions described herein are at a cell density of at least 1×10⁵ cells/mL, at least 2×10⁵ cells/mL, at least 4×10⁵ cells/mL, at least 6×10⁵ cells/mL, at least 8×10⁵ cells/mL, at least 0.5×10⁶ cells/mL, at least 1×10⁶ cells/mL, at least 2×10⁶ cells/mL, at least 4×10⁶ cells/mL, at least 6×10⁶ cells/mL, at least 8×10⁶ cells/mL, at least 10×10⁶ cells/mL, at least 12×10⁶ cells/mL, at least 14×10⁶ cells/mL, at least 16×10⁶ cells/mL, at least 18×10⁶ cells/mL, at least 20×10⁶ cells/mL, at least 22×10⁶ cells/mL, at least 24×10⁶ cells/mL, at least 26×10⁶ cells/mL, at least 28×10⁶ cells/mL, at least 30×10⁶ cells/mL, at least 32×10⁶ cells/mL, at least 34×10⁶ cells/mL, at least 36×10⁶ cells/mL, at least 38×10⁶ cells/mL, at least 40×10⁶ cells/mL, at least 42×10⁶ cells/mL, at least 44×10⁶ cells/mL, at least 46×10⁶ cells/mL, at least 48×10⁶ cells/mL, at least 50×10⁶ cells/mL, or at least 52×10⁶ cells/mL. In some embodiments, the cells are at a density of at least 12×10⁶ cells/mL or at least 24×10⁶ cells/mL.

In some aspects, a composition comprising about 12×10⁶ cells/mL, about 2.2 μg per million cells of one or more cationic polymers, about 10% of stabilizer relative to the one or more cationic polymers, and about 1 μg per million cells of one or more nucleic acids is provided.

In some aspects, a composition comprising about 24×10⁶ cells/mL, about 2.2 μg per million cells of one or more cationic polymers, about 25% of stabilizer relative to the one or more cationic polymers, and about 1 μg per million cells of one or more nucleic acids is provided.

The one or more cationic polymers, the stabilizer, the one or more nucleic acids and the cells included in the compositions of the present disclosure are as described herein. In some embodiments, the one or more cationic polymer comprises PEImax. In some embodiments, the stabilizer comprises comprises branched PEI with a molecular weight of about 25,000 Da grafted with PEG having a molecular weight of about 5,000 Da. In some embodiments, the one or more nucleic acids comprise (a) one or more plasmids comprising nucleic acids encoding AAV packaging proteins and/or nucleic acids encoding helper proteins, and/or (b) a plasmid comprising a nucleic acid encoding a transgene of interest. In some embodiments, the cells comprise HEK293 cells.

In some aspects, transfected cells produced using the methods described herein are provided. In some aspects, rAAV vectors produced using the methods described herein are provided.

The present disclosure also provides kits with packaging material and one or more components therein. A kit typically includes a label or packaging insert including a description of the components or instructions for use of the components therein. A kit can contain a collection of such components, e.g., a nucleic acid (plasmid), cationic polymer (e.g., PEI), stabilizer (e.g., bPEI25k.PEG5k, branched PEI with molecular weight (Mn) 25,000 conjugated to Polyethylene glycol (PEG) with Mₙ 5,000) and/or cells.

A kit refers to a physical structure housing one or more components of the kit. Packaging material can maintain the components in a sterile manner and can be made of material commonly used for such purposes (e.g., paper, corrugated fiber, glass, plastic, foil, ampules, vials, tubes, etc.).

Labels or inserts can include identifying information of one or more components therein. Labels or inserts can include information identifying manufacturer, lot numbers, manufacture location and date, expiration dates. Labels or inserts can include information identifying manufacturer information, lot numbers, manufacturer location and date. Labels or inserts can include instructions for using one or more of the kit components in a method, use, or manufacturing protocol. Instructions can include instructions for producing the compositions or practicing any of the methods described herein.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, magnetic tape, or an electrical storage media such as RAM and ROM or hybrids of these such as magnetic/optical storage media, FLASH media or memory type cards.

The following examples are offered for illustrative purposes only and are not intended to limit the scope of the present invention in any way. Indeed, various modifications of the invention in addition to those shown and described herein will become apparent to those skilled in the art from the foregoing description and fall within the scope of the appended claims.

### EXAMPLES

### Example 1. Stabilizing the DNA-PEI Complex by Spiking in Stabilizer (Branched PEI Conjugated to PEG

The aim of this study was to test if spiking the transfection cocktail with very low amount of PEG conjugated PEI (stabilizer) can abrogate particle size increase or aggregation of DNA-PEI complex.

### Materials and Methods:

1. Plasmid DNA (DNA): Three plasmids (plasmid encoding transgene of interest, a packaging plasmid encoding rep and cap genes, and an adenoviral helper plasmid encoding adenovirus E2, E4 and VARNA genes) capable of producing recombinant adeno-associated viral vectors (rAAV) were used in all the studies described herein.
2. Cationic polymer: Linear fully depropionylated polyethylenimine (PEI; 1 mg/ml stock concentration) 40 KDa, was used as transfection reagent.
3. Media: FreeStyle™ F17 (F17) expression medium (Thermo Fisher Scientific, A1383501 was used for making the transfection cocktail.
4. Stabilizer: Cationic polymer conjugated to neutral polymer was used as a stabilizer for these studies. Specifically, for this study we used bPEI25k.PEG5k (Sigma Aldrich, 900743; branched PEI with molecular weight (Mn) 25,000 conjugated to Polyethylene glycol (PEG) with Mₙ 5,000).
5. Spectrophotometry based turbidity test: Plasmid DNA (DNA) and PEI in F17 alone do not give any signal at 650 nm wavelength on nanodrop. However, when DNA and PEI are mixed together the culture turns turbid as the DNA and PEI complexes, and a signal is observed at 650 nm on nanodrop. With time, the continuous aggregation of DNA-PEI complexes lowers the turbidity and leads to a drop of nanodrop signal at 650 nm. This approach was used to test the stability of the DNA-PEI containing transfection cocktail with and without stabilizer.
6. Transfection cocktail preparation: The transfection cocktail consists of an equivolume mix containing: Mix A—Plasmid DNA (described above) added to F17 media in a predetermined ratio at 0.12 mg/ml final concentration and Mix B—0.264 mg/ml of PEI without or with 0.0264 mg/ml final concentration of stabilizer (this corresponds to 10% stabilizer relative to the cationic polymer PEI) in F17 media. The final transfection cocktail is prepared by adding Mix B to Mix A at room temperature (about 25° C.). Mixing is achieved by shaking the transfection cocktail continuously in a shaker at 120 RPM.
   Results: The transfection cocktail containing DNA and PEI was prepared in F17 media as described above in the Materials and Methods without and with stabilizer (bPEI25k.PEG5k). In addition, a solution containing DNA and stabilizer but without PEI was used as negative control. The three solutions were analyzed on nanodrop at 650 nm to test turbidity (FIG. 2). The transfection cocktail without stabilizer (square) gave a high signal for up to 10 minutes indicating formation of DNA-PEI complexes. However, after 10 minutes the signal quickly dropped to very low levels, reaching close to baseline levels by about 20 minutes. The transfection cocktail with stabilizer (circle) showed a lower signal than without stabilizer for the first about 10 minutes, but importantly retained the signal for later time points (at least up to 55 minutes). The negative control solution with DNA and stabilizer but without PEI (triangle) showed baseline signal suggesting no to very low complexation of stabilizer with DNA.
   Conclusion: The turbidity profile suggests that DNA and PEI formed complexes in the absence of stabilizer but started aggregating after about 10 minutes. In the presence of the stabilizer, however, DNA and PEI complex formation was stabilized (i.e., no aggregation) for at least the maximum time tested in this study (i.e., up to 55 minutes).

### Example 2. Impact of the Stabilizer (bPEI25k.PEG5k) on the Particle Size of DNA-PEI Complex

The aim of this study was to check the average particle size of the DNA-PEI complexes in the transfection cocktail in the presence of stabilizer (bPEI25k.PEG5k) Material and Methods:
1. Plasmid DNA, Cationic polymer, Media and Stabilizer: Same as described in Example 1.
2. Transfection cocktail preparation: The transfection cocktail consists of an equivolume mix containing: Mix A—Plasmid DNA (described in Example 1) added to F17 media in a predetermined ratio at 0.12 mg/ml final concentration and Mix B—0.264 mg/ml of PEI and 0, 0.0198, 0.0264, 0.03168 and 0.0396 mg/ml (which corresponds to 0%, 7.5%, 10%, 12.5% and 15% relative to the cationic polymer PEI) final concentration of stabilizer in F17 media. The final transfection cocktail is prepared by adding Mix B to Mix A at room temperature (about 25° C.).
3. Dynamic Light Scattering (DLS) experiment: The transfection cocktail with varying amounts of stabilizer (0 to 15% relative to PEI) was analyzed using DLS. Once the transfection cocktail was prepared, readings were taken every 5 minutes for 30 minutes.
   Results: FIG. 3 shows that the average particle size (nm) of the DNA-PEI complexes was reduced with increasing amount of the stabilizer.
   Conclusion: Stabilizer controls the particle size of the DNA-PEI complexes (FIG. 3), thereby inhibiting formation of large aggregates in the transfection cocktail.

### Example 3. Impact of the Stabilizer (bPEI25k.PEG5k) on the Transfection Efficiency of DNA-PEI Complex

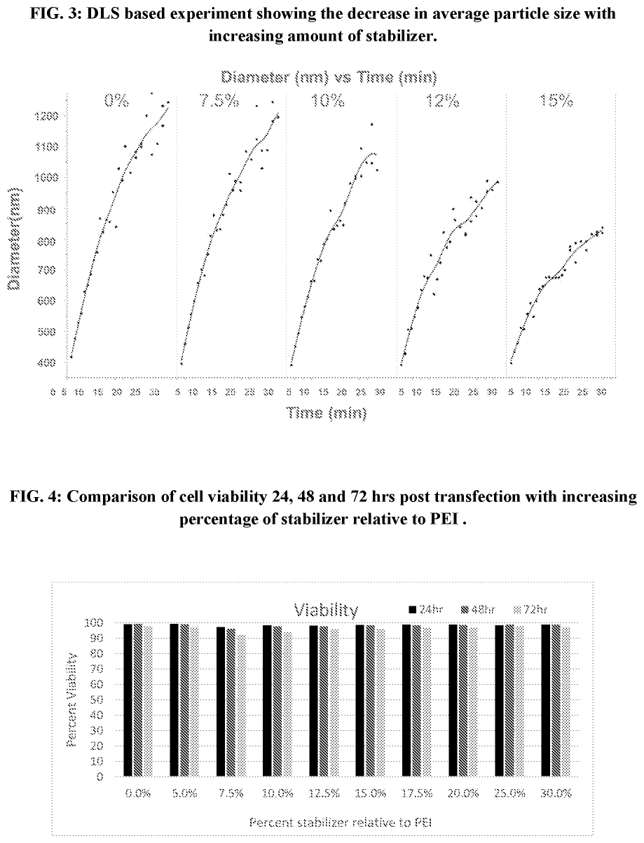

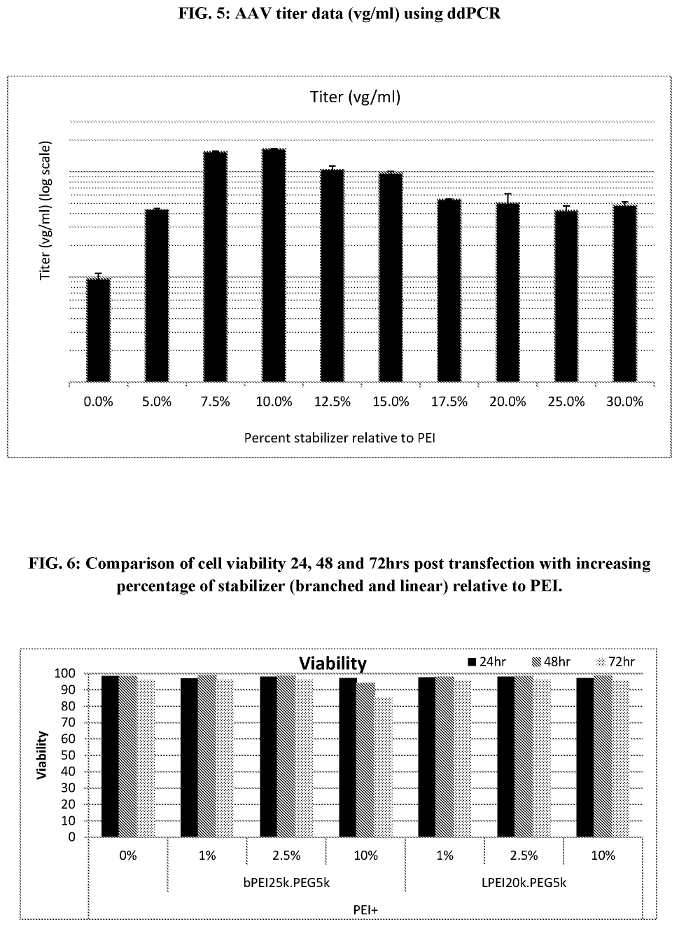

The aim of this study was to check the transfection efficiency of the transfection cocktail in the presence of stabilizer (bPEI25k.PEG5k) Material and Methods:
1. Plasmid DNA, Cationic polymer, Media and Stabilizer: Same as described in Example 1.
2. Transfection cocktail preparation: The transfection cocktail consists of an equivolume mix containing: Mix A—Plasmid DNA (as described in Example 1) added to F17 media in a predetermined ratio at 0.12 mg/ml final concentration and Mix B—0.264 mg/ml of PEI and 0, 0.0132, 0.0198, 0.0264, 0.03168, 0.0396, 0.04488, 0.0528, 0.066 and 0.0792 mg/ml final concentration of stabilizer (this corresponds to 0%, 5%, 7.5%, 10%, 12.5%, 15%, 17.5%, 20%, 25% and 30% relative to cationic polymer PEI) in F17 media. The final transfection cocktail is prepared by adding Mix B to Mix A at room temperature (about 25° C.). Mixing is achieved by shaking the transfection cocktail continuously in a shaker at 120 RPM. The transfection cocktail was 10% of the total cell culture volume and DNA amount used corresponds to 1 ug per million cells.
3. Transfection and AAV productivity test: HEK293 cells were grown at 37° C., 5% CO₂ in F17 media to a cell density of 12×10⁶ cells/mL in a six well plate. Prior to transfection, the transfection cocktail (prepared as described in (2) above) was incubated at room temperature for 1 h after preparation (“transfection cocktail incubation time”), and then added to the cell culture. Three hours post transfection, cultures were diluted to 1×10⁶ cells/mL to allow cell growth and AAV production. Samples, including cells and cell culture media, were taken at 24, 48 and 72 hours post transfection for cell count and cell viability (using Vi-cell). The cells were harvested at 72 hours post transfection, lysed and the lysate was analyzed for AAV titers using ddPCR.
   Results: It was found that the addition of stabilizer to the culture was not toxic to the cells as indicated by the high viability data shown in FIG. 4. Also, addition of stabilizer to the transfection cocktail increased AAV titer by at least 4-fold (FIG. 5). Highest level of rAAV production (about 10-fold increase) was obtained using about 7.5% to 10% of stabilizer (relative to cationic polymer, PEI.
   Conclusion: Use of stabilizer increased the transfection efficiency of the DNA-PEI complex as measured by rAAV titers. The optimal range of rAAV titer was obtained using about 7.5% to 15% stabilizer along with PEI in the transfection cocktail. While lower or higher amounts of stabilizer (relative to PEI) resulted in a drop of rAAV titer, addition of stabilizer increased rAAV titer as compared to that obtained in the absence of stabilizer.

### Example 4. Comparison of Linear Vs Branched PEI Conjugated to PEG as a Stabilizer

The aim of this study was to compare the ability of linear PEI to branched PEI conjugated to PEG in stabilizing the PEI-DNA transfection cocktail.

### Material and Methods:

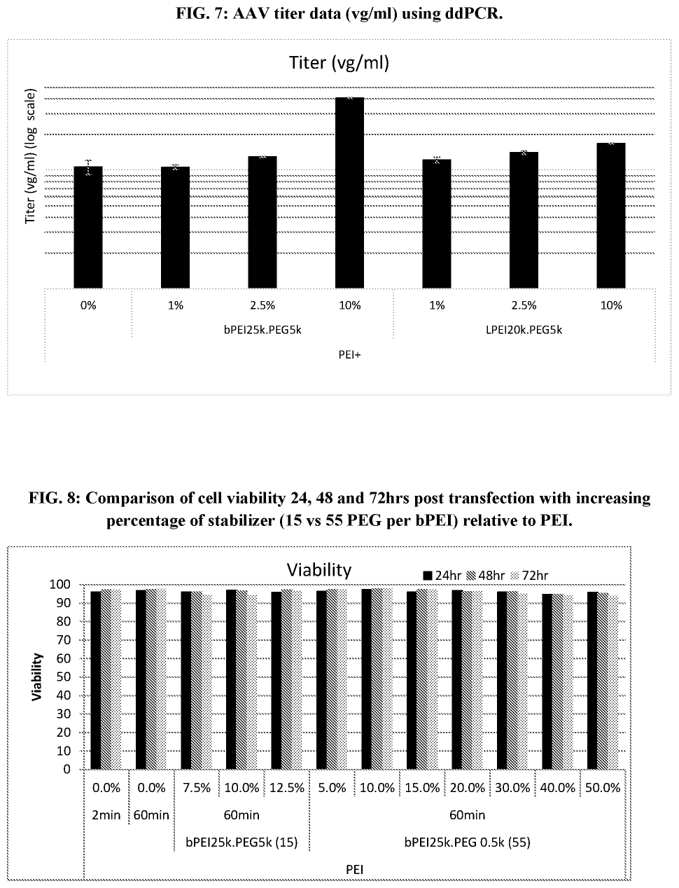

1. Plasmid DNA, Cationic polymer, and Media: Same as described in Example 1.
2. Stabilizer: For this study we used the following stabilizers: bPEI25k.PEG5k (branched PEI, Mₙ 25000 conjugated to Polyethylene glycol (PEG), Mₙ 5000 and LPEI20k.PEG5k (Linear PEI, Mₙ 20000 conjugated to PEG, Mₙ 5000)
3. Transfection cocktail preparation: The transfection cocktail consists of an equivolume mix containing: Mix A—Plasmid DNA (described in Example 1) added to F17 media in a predetermined ratio and Mix B—2.2 ug PEI per million cells and 0%, 1%, 2.5% and 10% of stabilizer (bPEI25k.PEG5k or LPEI20k.PEG5k) relative to PEI in F17 media. The final transfection cocktail is prepared by adding Mix B to Mix A at room temperature (about 25° C.). Mixing is achieved by shaking the transfection cocktail continuously in a shaker at 120 RPM. The transfection cocktail volume is 10 percent of the culture volume and DNA amount used corresponds to 1 ug per million cells.
4. Transfection and AAV productivity test: HEK293 cells were grown at 37° C., 5% CO₂ in F17 media to a cell density of 12×10⁶ cells/mL in a six well plate. Prior to transfection, the transfection cocktail (prepared as described in (3) above) was incubated at room temperature for 1 h after preparation (“transfection cocktail incubation time”), and then added to the cell culture. Three hours post transfection, cultures were diluted to 1×10⁶ cells/mL to allow cell growth and AAV production. Samples, including cells and cell culture media, were taken at 24, 48 and 72 hours post transfection for cell count and cell viability (using Vi-cell). The cells were harvested at 72 hours post transfection, lysed and the lysate was analyzed for AAV titers using ddPCR.
   Results: As described above, the cells were transfected with transfection cocktail containing 0% to 10% (relative to PEI) of either branched (bPEI25k.PEG5k) or linear (LPEI20k.PEG5k) stabilizer. It was found that both branched and linear stabilizers were not toxic to the cells (FIG. 6). Also, while addition of both stabilizers to the transfection cocktail increased the rAAV titer, the yield was higher with branched stabilizer as compared to linear stabilizer (FIG. 7). Highest level of rAAV production (four-fold increase) was obtained using about 10% of branched stabilizer (relative to cationic polymer, PEI).
   Conclusion: Both branched (bPEI25k.PEG5k) and linear (LPEI20k.PEG5k) stabilizers were not toxic to the cells and showed 2 to 4-fold titer improvement compared to the yield in the absence of stabilizer. While the sizes of the linear and branched stabilizer are comparable, the branched stabilizer gave higher rAAV titer as compared to linear stabilizer (four-fold improvement when 10% stabilizer is used relative to PEI).

### Example 5. Comparison of Number of PEG Molecules Conjugated Per Branched PEI

The aim of this study was to compare the stabilizing ability of PEGylated PEI compounds with different size and number of PEG molecules per branched PEI.

### Material and Methods:

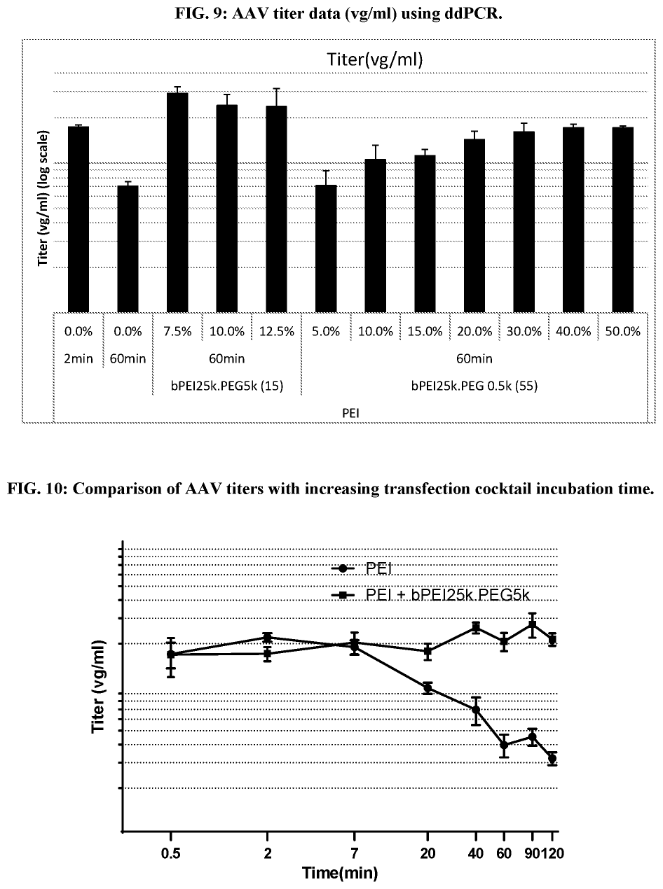

1. Plasmid DNA, Cationic polymer, and Media: Same as described in Example 1
2. Stabilizer: For this study, we used: (i) bPEI25k.PEG5k (branched PEI, Mₙ 25000 conjugated to PEG, Mₙ 5000). This stabilizer has 15 PEG molecules per bPEI. (ii) bPEI25k.PEG0.5k (branched PEI, Mₙ 25000, conjugated to PEG, Mₙ 500). This stabilizer has 55 PEG molecules per bPEI.
3. Transfection cocktail preparation: The transfection cocktail consists of an equivolume mix containing: Mix A—Plasmid DNA (described in Example 1) added to F17 media in a predetermined ratio and Mix B—2.2 ug PEI per million cells and 0%, 7.5%, or 12.5% of bPEI25k.PEG5k, or 5%, 10%, 15%, 20%, 30%, 40%, or 50% of bPEI25k.PEG0.5k relative to PEI in F17 media. The final transfection cocktail is prepared by adding Mix B to Mix A at room temperature (about 25° C.). Mixing is achieved by shaking the transfection cocktail continuously in a shaker at 120 RPM. The transfection cocktail volume is 10 percent of the culture volume and DNA amount used corresponds to 1 ug per million cells.
4. Transfection and AAV productivity test: HEK293 cells were grown at 37° C., 5% CO₂ in F17 media to a cell density of 12×10⁶ cells/mL in a six well plate. Prior to transfection, the transfection cocktail (prepared as described in (3) above) was incubated at room temperature for 1 h after preparation (“transfection cocktail incubation time”), and then added to the cell culture. Three hours post transfection, cultures were diluted to 1×10⁶ cells/mL to allow cell growth and AAV production. Samples, including cells and cell culture media, were taken at 24, 48 and 72 hours post transfection for cell count and cell viability (using Vi-cell). The cells were harvested at 72 hours post transfection, lysed and the lysate was analyzed for AAV titers using ddPCR.
   Results: As described above, the cells were transfected with transfection cocktail containing 0% to 12.5% of bPEI25k.PEG5k (with 15PEG per bPEI) or 0% to 50% of bPEI25k.PEG0.5k (55PEG per bPEI) stabilizer relative to PEI. The viability data (FIG. 8) suggested that addition of 15 or 55PEG per bPEI containing stabilizer to the culture was not toxic to the cells. Addition of bPEI25k.PEG5k (with 15PEG per bPEI) to the transfection cocktail increased rAAV titer by 4-fold, while addition of bPEI25k.PEG0.5k (55PEG per bPEI) increased rAAV titer by 2.4 fold as compared to the rAAV yield in the absence of stabilizer (FIG. 9).
   Conclusion: Both stabilizers (bPEI25k.PEG5k (with 15PEG per bPEI) and bPEI25k.PEG0.5k (with 55PEG per bPEI)) were not toxic to the cells and were able to stabilize the transfection cocktail as evidenced by the stable titer yield even after 60 minutes of transfection cocktail incubation time. Use of bPEI25k.PEG5k resulted in higher rAAV titer yield as compared to bPEI25k.PEG0.5k (4 versus 2.4-fold respectively).

### Example 6. Impact of Transfection Cocktail Incubation Time on AAV Titer

The aim of this study was to compare the impact of transfection cocktail incubation time with and without stabilizer (bPEI25k.PEG5k)

### Material and Methods:

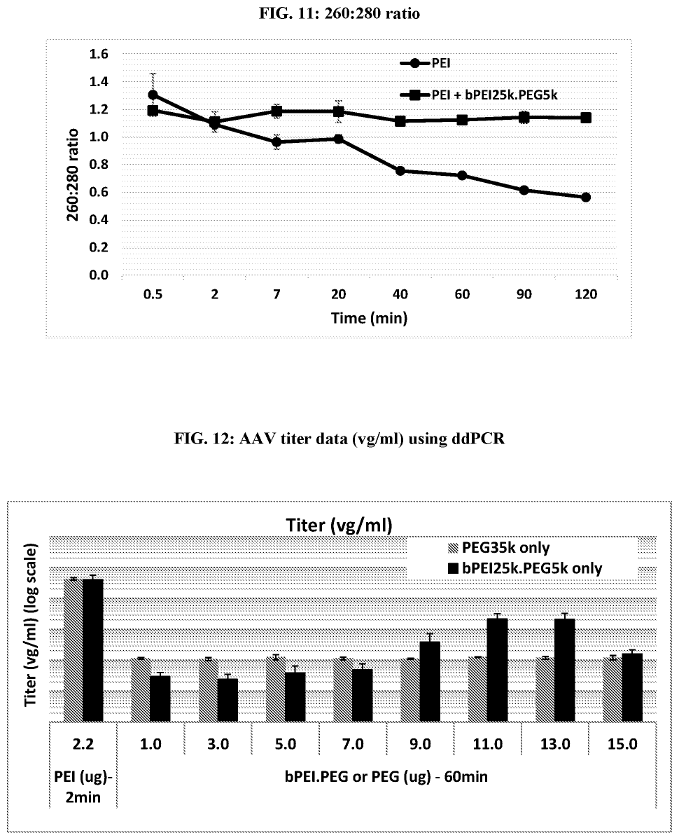

1. Plasmid DNA, Cationic polymer, Media and Stabilizer: Same as described in Example 1.
2. Transfection cocktail preparation: The transfection cocktail consists of an equivolume mix containing, Mix A—Plasmid DNA (described in Example 1) added to F17 media in a predetermined ratio and Mix B—2.2 ug PEI per million cells and either 0% or 10% of stabilizer (bPEI25k.PEG5k) relative to PEI in F17 media. The final transfection cocktail is prepared by adding Mix B to Mix A at room temperature (about 25° C.). Mixing is achieved by shaking the transfection cocktail continuously in a shaker at 120 RPM. The transfection cocktail volume is 10 percent of the culture volume and DNA amount used corresponds to 1 ug per million cells.
3. Transfection and AAV productivity test: HEK293 cells were grown at 37° C., 5% CO₂ in F17 media to a cell density of 12×10⁶ cells/mL in an Ambr®15 bioreactor. Prior to transfection, the transfection cocktail (prepared as described in (2) above) was incubated at room temperature for 30 seconds, 2, 7, 20, 40, 60, 90 and 120 minutes after preparation (“transfection cocktail incubation time”), and then added to the cell culture. Three hours post transfection, transfection was quenched. The cells were maintained in growth phase to allow AAV production. Samples, including cells and cell culture media, were taken at 24, 48 and 72 hours post transfection for cell count and cell viability (using Vi-cell). The cells were harvested at 72 hours post transfection, lysed and the lysate was analyzed for AAV titers using ddPCR.
4. Empty to full particle analysis: The cell lysate was purified using size exclusion high pressure liquid chromatography (SE-HPLC) and the elution peaks were compared between 260 nm and 280 nm to determine the ratio of empty to full vectors (See, for example, Sommer et al. (2003) Quantification of adeno-associated virus particles and empty capsids by optical density measurement. Molecular Therapy, Vol 7, Issue 1, P122-128). A comparison of the 260:280 ratio of empty, intermediate and full vectors revealed that the empty vectors have a 260:280 ratio close to 0.6, the intermediates have a 260:280 ratio between 0.9 and 1.1, while the full vectors have a 260:280 ratio close to 1.3 to 1.4.
   Results: As described above, the transfection cocktail containing either 0% or 10% of bPEI25k.PEG5k (relative to PEI) was prepared and incubated between 0.5 to 120 minutes before adding to the cell culture. For the first about 7 minutes, addition of the stabilizer had no impact on the rAAV titer (FIG. 10). However, after about 7 minutes, the amount rAAV produced in the absence of stabilizer decreased, while the amount of rAAV produced in the presence of stabilizer remained unaffected (FIG. 10). Thus, with stabilizer, rAAV titers were not affected by increase in the incubation time of the transfection cocktail for at least 120 minutes. 260:280 ratio, an indirect measure of full to empty AAV vectors (i.e., AAV particles carrying DNA versus AAV particles carrying no DNA) was also measured (FIG. 11). A continuous drop in 260:280 ratio was observed without stabilizer (i.e., transfection conducted in the presence of PEI alone) whereas a high ratio was maintained with stabilizer (i.e., transfection conducted using PEI and bPEI25k.PEG5k).
   Conclusion: This study demonstrates that the stabilizer can maintain both AAV titers and a higher number of full vectors as compared to empty vectors as evidenced by the 260:280 ratios (1.0 to 1.2) for the longest transfection cocktail incubation time tested in this experiment (i.e., 120 minutes). In the absence of stabilizer, rAAV titers dropped significantly (by about 7-8 fold) while the 260:280 ratio decreased to about 0.6 with increasing transfection cocktail incubation time indicating an increase in the number of empty vectors.

### Example 7. Transfection Efficiency of Stabilizer Alone in the Absence of PEI

The aim of this study was to determine the transfection efficiency of stabilizer when used alone (i.e., in the absence of cationic polymer) as a transfection reagent

### Material and Methods:

1. Plasmid DNA and Media: Same as described in Example 1.
2. Transfection reagent: For this study, we used either (i) PEI (as described in Example 1; positive control) (ii) bPEI25k.PEG5k (branched PEI, Mₙ 25000, conjugated to PEG, Mₙ 5000) or (iii) PEG Mₙ 35000 (PEG35k) as the transfection reagent.
3. Transfection cocktail preparation: The transfection cocktail consists of an equivolume mix containing: Mix A—Plasmid DNA (described in Example 1) added to F17 media in a predetermined ratio and Mix B—containing either (i) PEI (positive control; 2.2 ug per million cells), (ii) bPEI25k.PEG5k (1 to 15 ug per million cells) or (iii) PEG35k (1 to 15 ug per million cells). The final transfection cocktail is prepared by adding Mix B to Mix A at room temperature (about 25° C.). Mixing is achieved by shaking the transfection cocktail continuously in a shaker at 120 RPM. The transfection cocktail volume is 10 percent of the culture volume and DNA amount used corresponds to 1 ug per million cells.
5. Transfection and AAV productivity test: HEK293 cells were grown at 37° C., 5% CO₂ in F17 media to a cell density of 12×10⁶ cells/mL in a six well plate. Prior to transfection, the transfection cocktail containing PEI alone as the transfection reagent (prepared as described in (3) above) was incubated at room temperature for 2 minutes, while the transfection cocktail containing different amounts of either bPEI25k.PEG5k or PEG35k as the transfection reagent was incubated at room temperature for 1 h after preparation (“transfection cocktail incubation time”), and then added to the cell culture. Three hours post transfection, cultures were diluted to 1×10⁶ cells/mL to allow cell growth and AAV production. Samples, including cells and cell culture media, were taken at 24, 48 and 72 hours post transfection for cell count and cell viability (using Vi-cell). The cells were harvested at 72 hours post transfection, lysed and the lysate was analyzed for AAV titers using ddPCR.
   Results: To understand the role of PEG and bPEI in the transfection process, DNA transfections were done with increasing amount of either bPEI25k.PEG5k alone or PEG35k alone (after 60 minutes of transfection cocktail incubation time) and the transfection efficiency (measured as rAAV titer) was compared to PEI alone (2 minutes transfection cocktail incubation time) (FIG. 12). Use of PEG35k as the transfection reagent did not result in any increase in the rAAV titers whereas use of bPEI25k.PEG5k at a concentration of about 11 to 13 ug per million cells resulted in a maximum titer which was still about 10-fold lower than the positive control (i.e., PEI alone as the transfection reagent).
   Conclusion: This study demonstrates that bPEI25k.PEG5k interacts with the PEI-DNA complex via the branched PEI moiety (bPEI) and not by PEG molecule. PEG alone cannot stabilize or even transfect the cells. The combination of a PEG molecule conjugated to a cationic polymer such as PEI is key to stabilizing the transfection cocktail. Based on these studies, it is hypothesized that the PEI moiety of bPEI25k.PEG5k allows the stabilizer to bind to the DNA while the PEG molecule stabilizes the DNA-PEI complex by reducing the surface charge of DNA-PEI complexes and minimizing aggregate formation.

### Example 8. Transfection Efficiency of the Combination of Cationic Polymer and Stabilizer as Compared to Cationic Polymer Alone

The aim of this study was to determine the transfection efficiency of the combination of cationic polymer and stabilizer as compared to cationic polymer alone.

### Material and Methods:

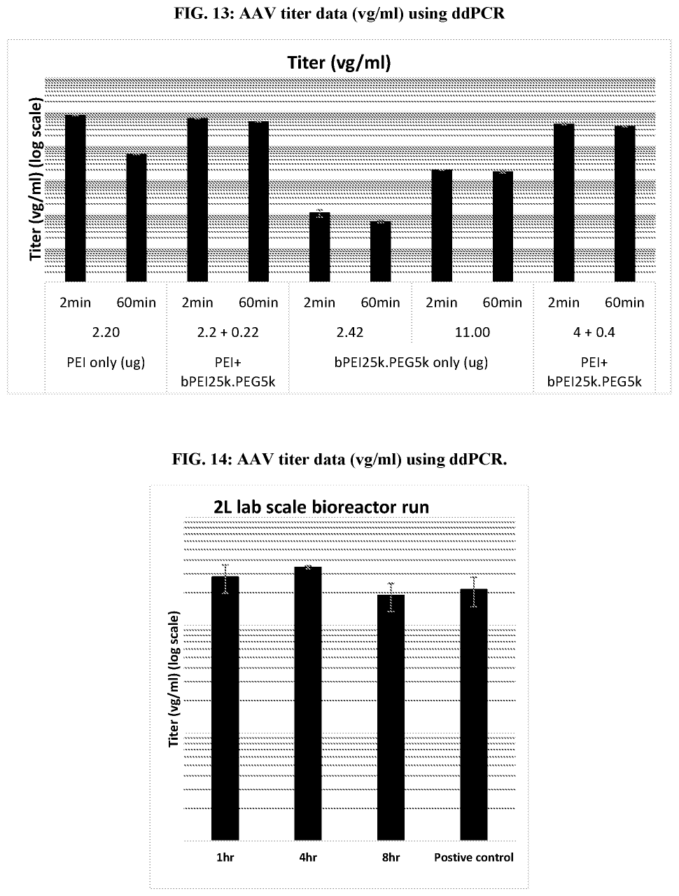

1. Plasmid DNA, Cationic Polymer, Media and Stabilizer: As described in Example 1.
2. Transfection cocktail preparation: The transfection cocktail consists of an equivolume mix containing: Mix A—Plasmid DNA (as described in Example 1) added to F17 media in a predetermined ratio and Mix B—containing (i) PEI (2.2 ug per million cells), (ii) PEI (2.2 ug per 5 million cells) and stabilizer (bPEI25k.PEG5k) (0.22 ug per million cells which corresponds to 10% relative to PEI), (iii) stabilizer (bPEI25k.PEG5k) (2.4 ug or 11 ug per million cells), or (iv) PEI (4 ug per million cells) and stabilizer (bPEI25k.PEG5k) (0.4 ug per million cells which corresponds to 10% relative to PEI). The final transfection cocktail is prepared by adding Mix B to Mix A at room temperature (about 25° C.). Mixing is achieved by shaking the transfection cocktail continuously in a shaker at 120 RPM. The transfection cocktail volume is 10 percent of the culture volume and DNA amount used corresponds to 1 ug per million cells.
3. Transfection and AAV productivity test: HEK293 cells were grown at 37° C., 5% CO₂ in F17 media to a cell density of 12×10⁶ cells/mL in a six well plate. Prior to transfection, the transfection cocktail (prepared as described in (2) above) was incubated at room temperature for 2 minutes, or 60 minutes (“transfection cocktail incubation time”), and then added to the cell culture. Three hours post transfection, cultures were diluted to 1×10⁶ cells/mL to allow cell growth and AAV production. Samples, including cells and cell culture media, were taken at 24, 48 and 72 hours post transfection for cell count and cell viability (using Vi-cell). The cells were harvested at 72 hours post transfection, lysed and the lysate was analyzed for AAV titers using ddPCR.
Results: When PEI alone was used as the transfection reagent, the rAAV titers dropped by about 10 fold when the transfection cocktail incubation time increased from 2 to 60 minutes (compare 2 mins to 60 mins: bar 1 versus 2, FIG. 13). In contrast, addition of 10% of bPEI25k.PEG25k to PEI maintained high titers between 2 minutes and 60 minutes (compare bars 3 and 4 to bars 1 and 2). When bPEI25k.PEG5k alone (no PEI) was used for transfection in an amount equivalent to the amount of PEI and bPEI25k.PEG5k used in previous condition (bars 5 and 6, FIG. 13) or in an amount that is 5-fold higher than the amount of PEI used alone in previous condition shown in bars 1 and 2 (bars 7 and 8, FIG. 13) the rAAV titers were more than 10-20 fold lower for both 2 minutes and 60 minutes (bars 5-8 as compared to bars 1 and 2, FIG. 16). This suggests that the stabilizer used by itself alone stabilizes the DNA-PEI complexes but the titers are very low as compared to PEI used alone. The unique combination of PEI and stabilizer can stabilize the DNA-PEI complexes in the transfection cocktail and results in high titers. Finally, it was determined that if the PEI amount is also altered, whilst keeping stabilizer level at 10% relative to PEI, the transfection cocktail remained stable and resulted in high titers (bars 9-10, FIG. 13).
Conclusion: The transfection efficiency of stabilizer alone (i.e., in the absence of cationic polymer, PEI) is very poor (15-20 fold lower) as compared to PEI. The titer drops significantly (more than 10-15 fold) in the absence of stabilizer if the transfection cocktail is incubated for a long time (greater than 7-10 minutes). The combination of PEI with stabilizer helps to stabilize the DNA-PEI complexes and results in high rAAV titers.

### Example 9. Scalability and Long-Term Stability of the Transfection Cocktail

The aim of this study was to test the scalability of the transfection cocktail and prolonged transfection cocktail incubation time.

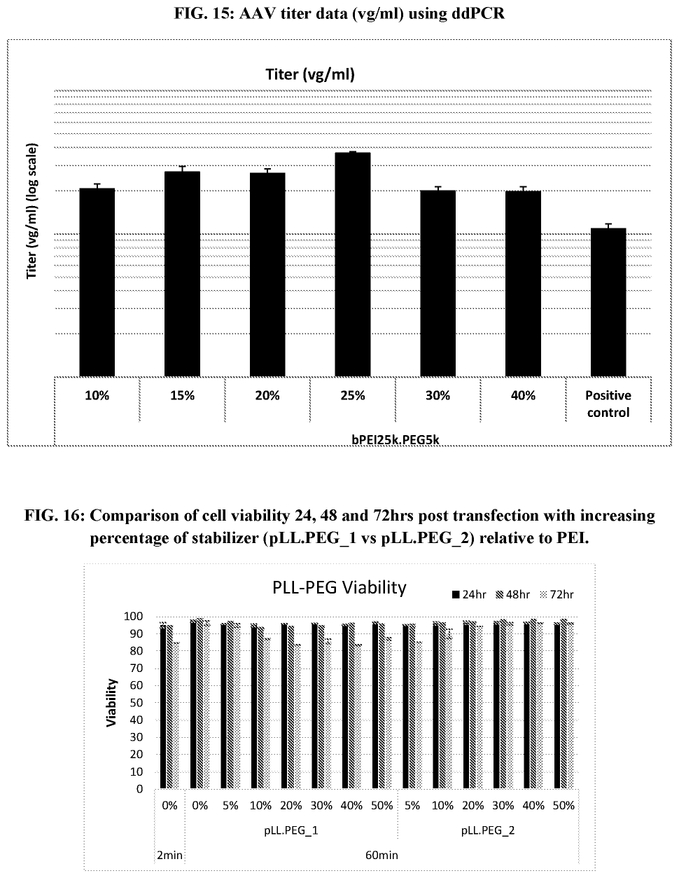

Material and Methods:
1. Plasmid DNA, Cationic Polymer, Media and Stabilizer: As described in Example 1.
2. Transfection cocktail preparation: The transfection cocktail consists of an equivolume mix containing: Mix A—Plasmid DNA (as described in Example 1) added to F17 media in a predetermined ratio and Mix B—2.2 ug PEI per million cells and 10% of stabilizer (bPEI25k.PEG5k) relative to PEI in F17 media. The final transfection cocktail is prepared by adding Mix B to Mix A at room temperature (about 25° C.). Mixing is achieved by shaking the transfection cocktail continuously in a shaker at 120 RPM. The transfection cocktail volume is 20 percent of the culture volume and DNA amount is 1 ug per million cells.
3. Transfection and AAV productivity test: HEK293 cells were grown at 37° C., 5% CO₂ in F17 media to a cell density of 12×10⁶ cells/mL in a 2 L bioreactor. Prior to transfection, the transfection cocktail (prepared as described in (2) above) was incubated at room temperature for 1, 4 or 8 hours (“transfection cocktail incubation time”), and then added to the cell culture. Three hours post transfection, transfection was quenched. The cells were maintained in growth phase to allow AAV production for 72 hours. Samples, including cells and cell culture media, were taken at 24, 48 and 72 hours post transfection for cell count and cell viability (using Vi-cell). The cells were harvested at 72 hours post transfection, lysed and the lysate was analyzed for AAV titers using ddPCR.
   Results: Irrespective of the transfection cocktail incubation time (1, 4 or 8 hours), comparable rAAV titers were obtained (FIG. 14).
   Conclusion: This study demonstrate that the transfection cocktail is stable for at least up to 8 hours and results in high rAAV titers.

### Example 10. Stabilizing Transfection Cocktail for Higher Cells Density

The aim of this study was to test the stability of the transfection cocktail containing stabilizer for high cell density transfections (24×10⁶ cells/mL).

### Material and Methods:

1. Plasmid DNA, Cationic Polymer, Media and Stabilizer: As described in Example 1.
2. Transfection cocktail preparation: The transfection cocktail consists of an equivolume mix containing: Mix A—Plasmid DNA (as described in Example 1) added to F17 media in a predetermined ratio and 10%, 20%, 30%, 40% or 50% of bPEI25k.PEG5k relative to PEI amount, and Mix B—2.2 ug PEI per million cells in F17 media. The final transfection cocktail is prepared by adding Mix B to Mix A at room temperature (about 25° C.). Mixing is achieved by shaking the transfection cocktail continuously in a shaker at 120 RPM. The transfection cocktail volume is 10 percent of the culture volume and DNA amount used corresponds to 1 ug per million cells.
3. Transfection and AAV productivity test: HEK293 cells were grown at 37° C., 5% CO₂ in F17 media to a cell density of 24×10⁶ cells/mL in a six well plate. Prior to transfection, the transfection cocktail (prepared as described in (2) above) was incubated at room temperature for 1 hour (“transfection cocktail incubation time”), and then added to the cell culture. Three hours post transfection, cultures were diluted to 2×10⁶ cells/mL to allow cell growth and AAV production. Samples, including cells and cell culture media, were taken at 24, 48 and 72 hours post transfection for cell count and cell viability (using Vi-cell). The cells were harvested at 72 hours post transfection, lysed and the lysate was analyzed for AAV titers using ddPCR.
   Results: At high cell density (24×10⁶ cells/mL), addition of stabilizer increased rAAV titer at all amounts tested as compared to positive control (FIG. 15). Highest rAAV titer levels were obtained in the presence of about 25% (relative to PEI) stabilizer (FIG. 15).
   Conclusion: This study demonstrates that for high cell density (e.g., 24×10⁶ cells/mL) transfections, the transfection cocktail (containing twice the concentration of DNA as used in Examples 1-9) can be stabilized using the stabilizer bPEI25k.PEG5k, with highest rAAV titer levels obtained using about 25% of stabilizer relative to PEI. Also, the order of addition of stabilizer in the transfection cocktail was found to be important. For high cell density (e.g., 24×10⁶ cells/mL) transfections, it is important to add the stabilizer to Mix A containing the plasmids, while for low cell density (e.g., 12×10⁶ cells/mL) transfections, the order of addition of stabilizer was immaterial (i.e., the stabilizer can be added to Mix A (containing the plasmid DNA) or to Mix B (containing PEI)).

### Example 11. Stabilizing the DNA-PEI Complex Using Alternate Stabilizer, PEG Conjugated to Poly L-Lysine (pLL.PEG)

The aim of this study was to compare the stabilizing ability of PEGylated poly L-Lysine (pLL) compounds.

### Material and Methods:

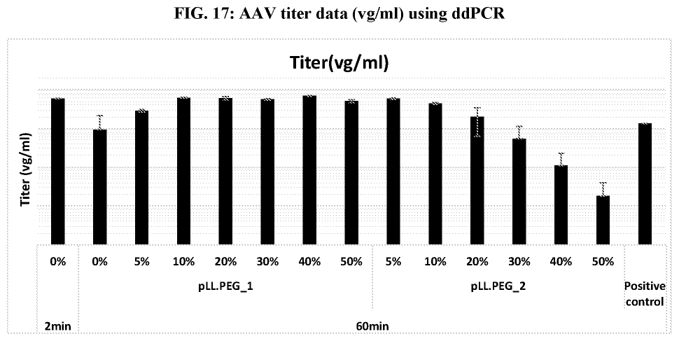

1. Plasmid DNA, Cationic polymer, and Media: Same as described in Example 1
2. Stabilizer: For this study, we used: (i) pLL.PEG_1 (pLL, Mₙ 26000, conjugated to PEG, Mₙ 5000). (ii) pLL.PEG_2 (pLL, Mₙ 15000, conjugated to PEG, Mₙ 5000)
3. Transfection cocktail preparation: The transfection cocktail consists of an equivolume mix containing: Mix A—Plasmid DNA (described in Example 1) added to F17 media in a predetermined ratio and Mix B—2.2 ug PEI per million cells and 0%, 5%, 10%, 20%, 30%, 40% or 50% of either pLL.PEG_1 or pLL.PEG_2 relative to PEI in F17 media. The final transfection cocktail is prepared by adding Mix B to Mix A at room temperature (about 25° C.). Mixing is achieved by shaking the transfection cocktail continuously in a shaker at 120 RPM. The transfection cocktail volume is 10 percent of the culture volume and DNA amount used corresponds to 1 ug per million cells.
4. Transfection and AAV productivity test: HEK293 cells were grown at 37° C., 5% CO₂ in F17 media to a cell density of 12×10⁶ cells/mL in a six well plate. Prior to transfection, the transfection cocktail (prepared as described in (3) above) was incubated at room temperature for 1 h after preparation (“transfection cocktail incubation time”), and then added to the cell culture. Three hours post transfection, cultures were diluted to 1×10⁶ cells/mL to allow cell growth and AAV production. Samples, including cells and cell culture media, were taken at 24, 48 and 72 hours post transfection for cell count and cell viability (using Vi-cell). The cells were harvested at 72 hours post transfection, lysed and the lysate was analyzed for AAV titers using ddPCR.
   Results: As described above, the cells were transfected with transfection cocktail containing 0% to 50% of pLL.PEG_1 or 0% to 50% of pLL.PEG_2 stabilizer relative to PEI. The viability data (FIG. 16) suggested that addition of pLL.PEG_1 or pLL.PEG_2 containing stabilizer to the culture was not toxic to the cells. Addition of pLL.PEG_1 or pLL.PEG_2 to the transfection cocktail increased rAAV titer by 6-fold (FIG. 17).
   Conclusion: While both stabilizers pLL.PEG_1 and pLL.PEG_2 were not toxic to the cells, use of pLL.PEG_1 and pLL.PEG_2 resulted in higher rAAV titer yield as compared to no stabilizer condition.

The various features and embodiments of the present invention, referred to in individual sections above apply, as appropriate, to other sections, mutatis mutandis. Consequently features specified in one section may be combined with features specified in other sections, as appropriate. All references cited herein, including patents, patent applications, papers, text books, and cited sequence Accession numbers, and the references cited therein are hereby incorporated by reference in their entirety. In the event that one or more of the incorporated literature and similar materials differs from or contradicts this application, including but not limited to defined terms, term usage, described techniques, or the like, this application controls.

We claim:

1. A method for transfecting cells with one or more nucleic acids, comprising the following steps:
(i) preparing a transfection cocktail comprising one or more cationic polymers, a stabilizer and one or more nucleic acids,
(ii) contacting the transfection cocktail prepared in step (i) with cells to be transfected to form a mixture, and
(iii) incubating the mixture of step (ii) thereby transfecting the cells with the one or more nucleic acids.

2. A method for making cells that produce recombinant adeno-associated viral (rAAV) vector, comprising the following steps:
(i) preparing a transfection cocktail comprising one or more cationic polymers, a stabilizer and one or more nucleic acids,
(ii) contacting the transfection cocktail prepared in step (i) with cells to be transfected to form a mixture, and
(iii) incubating the mixture of step (ii) thereby making transfected cells that produce rAAV vector.

3. A method for increasing transfection of cells with one or more nucleic acids, comprising the following steps:
(i) preparing a transfection cocktail comprising one or more cationic polymers, a stabilizer and one or more nucleic acids,
(ii) contacting the transfection cocktail prepared in step (i) with cells to be transfected to form a mixture, and
(iii) incubating the mixture of step (ii), whereby transfection of the cells with the one or more nucleic acids is increased as compared to the transfection of the cells performed under the same conditions but in the absence of the stabilizer.

4. A method for producing high titer recombinant adeno-associated viral (rAAV) vector, comprising the following steps:
(i) preparing a transfection cocktail comprising one or more cationic polymers, a stabilizer and one or more nucleic acids,
(ii) contacting the transfection cocktail prepared in step (i) with cells to be transfected to form a mixture,
(iii) incubating the mixture of step (ii) to make transfected cells that produce rAAV vector, and
(iv) isolating and/or purifying rAAV vector from the transfected cells produced in step (iii), wherein the time between initiation of step (i) and completion of step (ii) is greater than 10 seconds, and wherein the rAAV titer is increased by at least 2-fold and/or by at least 5% as compared to the rAAV titer produced under the same conditions but in the absence of the stabilizer.

5. The method of any one of claims 1-4, wherein the time between initiation of step (i) and completion of step (ii) is between about 10 seconds to about 10 days.

6. The method of any one of claims 1-5, wherein the time between initiation of step (i) and completion of step (ii) is more than 10 minutes.

7. The method of any one of claims 1-6, wherein the transfection cocktail of step (i) is incubated from about 10 seconds to about 10 days, about 15 seconds to about 5 days, about 30 seconds to about 2 days, about 60 seconds to about 1 day, about 90 seconds to about 10 hours, about 90 seconds to about 8 hours, about 2 minutes to about 4 hours, about 4 minutes to about 2 hours, about 6 minutes to about 1 hour, or about 10 minutes to about 30 minutes prior to step (ii).

8. The method of any one of claims 1-7, wherein the transfection cocktail is prepared by first mixing the one or more cationic polymers with the stabilizer to form a resultant mixture which is then added to the one or more nucleic acids.

9. The method of any one of claims 1-7, wherein the transfection cocktail is prepared by first mixing the stabilizer with the one or more nucleic acids to form a resultant mixture which is then added to the one or more cationic polymers.

10. The method of any one of claims 1-7, wherein the transfection cocktail is prepared by first mixing the one or more cationic polymers with the one or more nucleic acids to form a resultant mixture which is then added to the stabilizer.

11. The method of any one of claims 1-10, wherein the cells are at a cell density of at least 1×10⁵ cells/mL, at least 2×10⁵ cells/mL, at least 4×10⁵ cells/mL, at least 6×10⁵ cells/mL, at least 8×10⁵ cells/mL, at least 0.5×10⁶ cells/mL, at least 1×10⁶ cells/mL, at least 2×10⁶ cells/mL, at least 4×10⁶ cells/mL, at least 6×10⁶ cells/mL, at least 8×10⁶ cells/mL, at least 10×10⁶ cells/mL, at least 12×10⁶ cells/mL, at least 14×10⁶ cells/mL, at least 16×10⁶ cells/mL, at least 18×10⁶ cells/mL, at least 20×10⁶ cells/mL, at least 22×10⁶ cells/mL, at least 24×10⁶ cells/mL, at least 26×10⁶ cells/mL, at least 28×10⁶ cells/mL, at least 30×10⁶ cells/mL, at least 32×10⁶ cells/mL, at least 34×10⁶ cells/mL, at least 36×10⁶ cells/mL, at least 38×10⁶ cells/mL, at least 40×10⁶ cells/mL, at least 42×10⁶ cells/mL, at least 44×10⁶ cells/mL, at least 46×10⁶ cells/mL, at least 48×10⁶ cells/mL, at least 50×10⁶ cells/mL, or at least 52×10⁶ cells/mL when contacted with the transfection cocktail in step (ii).

12. The method of any one of claims 1-11, wherein the one or more cationic polymers are used in an amount of about 0.05 μg per million cells, about 0.1 μg per million cells, about 0.2 μg per million cells, about 0.4 μg per million cells, about 0.6 μg per million cells, about 0.8 μg per million cells, about 1.0 μg per million cells, about 1.5 μg per million cells, about 2 μg per million cells, about 2.1 μg per million cells, about 2.2 μg per million cells, about 2.3 μg per million cells, about 2.4 μg per million cells, about 2.5 μg per million cells, about 2.6 μg per million cells, about 2.7 μg per million cells, about 2.8 μg per million cells, about 2.9 μg per million cells, about 3.0 μg per million cells, about 3.5 μg per million cells, about 4.0 μg per million cells, about 4.5 μg per million cells, about 5 μg per million cells, about 5.5 μg per million cells, about 6.0 μg per million cells, about 6.5 μg per million cells, about 7.0 μg per million cells, about 7.5 μg per million cells, about 8.0 μg per million cells, about 10 μg per million cells.

13. The method of any one of claims 1-12 wherein the amount of stabilizer in the transfection cocktail is about 1%, about 2%, about 2.5%, about 5%, about 7.5%, about 10%, about 12.5%, about 15%, about 17.5%, about 20%, about 25%, about 30%, about 40%, about 50%, about 60%, about 70%, about 75%, about 80%, about 90%, about 100%, about 200%, about 300%, about 400%, or about 500% relative to the amount of the one or more cationic polymers.

14. The method of claim 13, wherein the cells are at a low cell density (e.g., less than about 18×10⁶ cells/mL) when contacted with the transfection cocktail in step (ii) and the amount of stabilizer in the transfection cocktail is about 7.5% to about 10% relative to the amount of the one or more cationic polymers.

15. The method of claim 13, wherein the cells are at a high cell density (e.g., more than about 18×10⁶ cells/mL) when contacted with the transfection cocktail in step (ii) and the amount of stabilizer in the transfection cocktail is about 15% to about 30% relative to the amount of the one or more cationic polymers.

16. The method of any one of claims 1-15, wherein the one or more nucleic acids are used in an amount of about 0.05 μg per million cells, about 0.1 μg per million cells, about 0.2 μg per million cells, about 0.4 μg per million cells, about 0.5 μg per million cells, about 0.6 μg per million cells, about 0.8 μg per million cells, about 1.0 μg per million cells, about 1.5 μg per million cells, about 2.0 μg per million cells, about 2.5 μg per million cells, about 3.0 μg per million cells, about 3.5 μg per million cells, about 4.0 μg per million cells, about 4.5 μg per million cells, about 5 μg per million cells, about 5.5 μg per million cells, about 6.0 μg per million cells, about 6.5 μg per million cells, about 7.0 μg per million cells, about 7.5 μg per million cells, about 8.0 μg per million cells, about 8.5 μg per million cells, about 9.0 μg per million cells, about 9.5 μg per million cells, or about 10 μg per million cells.

17. The method of any one of claims 1-16, wherein the mixture in step (iii) is incubated for about 15 minutes to about 150 hours to make transfected cells.

18. The method of any one of claims 1-17, wherein the one or more cationic polymers is selected from the group consisting of chitosan, poly-L-lysine, polyamine (PA), polyalkylenimine (PAI), polyethylenimine (PEI), poly[a-(-aminobutyl)-L-glycolic acid], chitosan, polyamidoamine, and poly(2-dimethylamino)ethyl methacrylate.

19. The method of claim 18, wherein the cationic polymer is branched or linear.

20. The method of claim 19, wherein the cationic polymer has a molecular weight ranging from about 500 to about 160,000 Da and/or about 2,500 to about 250,000 Da in free base form.

21. The method of claim 20, wherein the one or more cationic polymers is polyethylenimine (PEI).

22. The method of claim 21, wherein the cationic polymer comprises hydrolyzed linear PEI with a molecular weight of about 40,000 and/or about 22,000 molecular weight in free base form.

23. The method of any one of claims 1-22, wherein the stabilizer comprises a cationic polymer grafted with a poly(ethylene glycol) (PEG) moiety.

24. The method of claim 23, wherein PEG has a molecular weight from about 250 Da to 35,000 Da.

25. The method of claim 24, wherein the stabilizer comprises branched PEI with a molecular weight from about 2,000 to about 60,000 grafted with PEG having a molecular weight from about 250 Da to 35,000 Da.

26. The method of claim 25, wherein the stabilizer comprises branched PEI with a molecular weight of about 25,000 Da grafted with PEG having a molecular weight of about 5,000 Da.

27. The method of claim 26, wherein the number of PEG moieties grafted per molecule of cationic polymer is 1, 5, 10, 15, 20, 25, 30, 35, 40, 45, 50, 55, 60, 65, 70, 75, 80, 85, 90, 95, 100 or more.

28. The method of any one of claims 1-27, wherein the one or more nucleic acid molecules comprise (a) one or more plasmids comprising nucleic acids encoding AAV packaging proteins and/or nucleic acids encoding helper proteins, and/or (b) a plasmid comprising a nucleic acid encoding a transgene of interest.

29. The method of claim 28, wherein the one or more plasmids of (a) comprise a first plasmid comprising the nucleic acids encoding AAV packaging proteins and a second plasmid comprising the nucleic acids encoding helper proteins.

30. The method of any one of claims 28-29, wherein the encoded AAV packaging proteins comprise AAV rep and/or AAV cap proteins.

31. The method of any one of claims 28-30, wherein the encoded helper proteins comprise adenovirus E2 and/or E4, VARNA proteins, and/or non-AAV helper proteins.

32. The method of any one of claims 28-31, wherein the transgene encodes a wild type or functional variant blood clotting factor, mini-dystrophin, C1 esterase inhibitor, copper transporting P-type ATPase (ATP7B), or copper-zinc superoxide dismutase 1 (SOD1).

33. The method of any one of claims 1-3, 5-32 further comprising step (iv) isolating and/or purifying rAAV vector from the transfected cells produced in step (iii).

34. The method of any one of claims 1-33, wherein transfection of the cells with the one or more nucleic acids is increased by at least 5%, at least 10%, at least 15%, at least 20%, at least 25%, at least 30%, at least 35%, at least 35%, at least 40%, at least 45%, or at least 50% as compared to the transfection of the cells performed under the same conditions but in the absence of the stabilizer.

35. The method of any one of claims 1-34, wherein the amount of rAAV vector isolated/purified from the transfected cells is increased by at least 5%, at least 10%, at least 15%, at least 20%, at least 25%, at least 30%, at least 35%, at least 35%, at least 40%, at least 45%, or at least 50% or greater as compared to the amount of rAAV vector isolated/purified from the transfected cells under the same conditions but in the absence of the stabilizer.

36. The method of any one of claims 1-34, wherein the amount of rAAV vector isolated/purified from the transfected cells is increased by at least 2-fold, at least 4-fold, at least 10-fold, at least 20-fold, at least 40-fold, at least 50-fold, at least 60-fold, at least 80-fold, at least 100-fold or greater as compared to the amount of rAAV vector isolated/purified from the transfected cells under the same conditions but in the absence of the stabilizer.

37. The method of any one of claims 1-34, wherein the rAAV titer is increased by at least 2-fold at least 4-fold, at least 10-fold, at least 20-fold, at least 40-fold, at least 50-fold, at least 60-fold, at least 80-fold, at least 100-fold or greater as compared to the rAAV titer produced under the same conditions but in the absence of the stabilizer.

38. The method of any one of claims 1-34, wherein the rAAV titer is increased by at least 5%, at least 10%, at least 15%, at least 20%, at least 25%, at least 30%, at least 35%, at least 35%, at least 40%, at least 45%, or at least 50% or greater as compared to the rAAV titer produced under the same conditions but in the absence of the stabilizer.

39. The method of any one of claims 1-38, wherein the cells comprise mammalian cells, yeast cells, or insect cells.

40. The method of claim 39, wherein the cells are human embryonic kidney (HEK), Chinese hamster ovary (CHO) cells or insect-derived Sf9 cells.

41. The method of claim 40, wherein the cells are HEK 293E, HEK 293F or HEK 293T cells.

42. The method of claim 41, wherein the HEK 293 cells are adapted for serum-free growth in suspension.

43. The method of any one of claims 1-42, wherein the cells are stably or transiently transfected.

44. The method of any one of claims 1-43, wherein the cells are in suspension culture or are adherent.

45. The method of any one of claims 1-44, wherein the cells are grown or maintained in roller bottles or expanded roller bottles.

46. The method of any one of claims 1-44, wherein the cells are grown in bioreactors (e.g., WAVE bioreactor, stirred tank bioreactor), bags or flasks.

47. A composition comprising one or more cationic polymers and a stabilizer.

48. The composition of claim 49, further comprising one or more nucleic acids and/or cells.

49. A composition comprising a stabilizer and one or more nucleic acids.

50. The composition of claim 49, further comprising one or more cationic polymers and/or cells.

51. A composition comprising one or more cationic polymers, a stabilizer, one or more nucleic acids, and cells.

52. The composition of any one of claims 47-48, 50-51, wherein the amount of one or more cationic polymers is about 0.01 μg, about 0.02 μg, about 0.04 μg, about 0.06 μg, about 0.08 μg, about 1.0 μg, about 1.2 μg, about 1.4 μg, about 1.6 μg, about 1.8 μg, about 2.0 μg, about 2.2 μg, about 2.4 μg, about 2.6 μg, about 2.8 μg, about 3.0 μg, about 3.2 μg, about 3.4 μg, about 3.6 μg, about 3.8 μg, about 4.0 μg, about 4.2 μg, about 4.6 μg, about 4.8 μg, about 5.0 μg, about 5.5 μg, about 6.0 μg, about 6.5 μg, about 7 μg, about 7.5 μg, about 8.0 μg, about 8.5 μg, about 9.0 μg, about 9.5 μg, about 10 μg, about 12 μg, about 14 μg, about 16 μg, about 18 μg, about 20 μg, about 40 μg, about 60 μg, about 80 μg, about 100 μg, about 0.1 mg, about 1 mg, about 2 mg, about 4 mg, about 6 mg, about 8 mg or about 10 mg.

53. The composition of any one of claims 47-52, wherein the amount of stabilizer is about 0.01 μg, about 0.02 μg, about 0.04 μg, about 0.06 μg, about 0.08 μg, about 1.0 μg, about 1.2 μg, about 1.4 μg, about 1.6 μg, about 1.8 μg, about 2.0 μg, about 2.2 μg, about 2.4 μg, about 2.6 μg, about 2.8 μg, about 3.0 μg, about 3.2 μg, about 3.4 μg, about 3.6 μg, about 3.8 μg, about 4.0 μg, about 4.2 μg, about 4.6 μg, about 4.8 μg, about 5.0 μg, about 5.5 μg, about 6.0 μg, about 6.5 μg, about 7 μg, about 7.5 μg, about 8.0 μg, about 8.5 μg, about 9.0 μg, about 9.5 μg, about 10 μg, about 12 μg, about 14 μg, about 16 μg, about 18 μg, about 20 μg, about 40 μg, about 60 μg, about 80 μg, about 100 μg, about 0.1 mg, about 1 mg, about 2 mg, about 4 mg, about 6 mg, about 8 mg or about 10 mg.

54. The composition of any one of claims 48-53, wherein the amount of one or more nucleic acids is about 0.01 μg, about 0.02 μg, about 0.04 μg, about 0.06 μg, about 0.08 μg, about 1.0 μg, about 1.2 μg, about 1.4 μg, about 1.6 μg, about 1.8 μg, about 2.0 μg, about 2.2 μg, about 2.4 μg, about 2.6 μg, about 2.8 μg, about 3.0 μg, about 3.2 μg, about 3.4 μg, about 3.6 μg, about 3.8 μg, about 4.0 μg, about 4.2 μg, about 4.6 μg, about 4.8 μg, about 5.0 μg, about 5.5 μg, about 6.0 μg, about 6.5 μg, about 7 μg, about 7.5 μg, about 8.0 μg, about 8.5 μg, about 9.0 μg, about 9.5 μg, about 10 μg, about 12 μg, about 14 μg, about 16 μg, about 18 μg, about 20 μg, about 40 μg, about 60 μg, about 80 μg, about 100 μg, about 0.1 mg, about 1 mg, about 2 mg, about 4 mg, about 6 mg, about 8 mg or about 10 mg.

55. The composition of any one of claims 48, 50-54, wherein the cells are at a cell density of at least 1×10⁵ cells/mL, at least 2×10⁵ cells/mL, at least 4×10⁵ cells/mL, at least 6×10⁵ cells/mL, at least 8×10⁵ cells/mL, at least 0.5×10⁶ cells/mL, at least 1×10⁶ cells/mL, at least 2×10⁶ cells/mL, at least 4×10⁶ cells/mL, at least 6×10⁶ cells/mL, at least 8×10⁶ cells/mL, at least 10×10⁶ cells/mL, at least 12×10⁶ cells/mL, at least 14×10⁶ cells/mL, at least 16×10⁶ cells/mL, at least 18×10⁶ cells/mL, at least 20×10⁶ cells/mL, at least 22×10⁶ cells/mL, at least 24×10⁶ cells/mL, at least 26×10⁶ cells/mL, at least 28×10⁶ cells/mL, at least 30×10⁶ cells/mL, at least 32×10⁶ cells/mL, at least 34×10⁶ cells/mL, at least 36×10⁶ cells/mL, at least 38×10⁶ cells/mL, at least 40×10⁶ cells/mL, at least 42×10⁶ cells/mL, at least 44×10⁶ cells/mL, at least 46×10⁶ cells/mL, at least 48×10⁶ cells/mL, at least 50×10⁶ cells/mL, or at least 52×10⁶ cells/mL.

56. A composition comprising about 12×10⁶ cells/mL, about 2.2 μg per million cells of one or more cationic polymers, about 10% of stabilizer relative to the one or more cationic polymers, and about 1 μg per million cells of one or more nucleic acids.

57. A composition comprising about 24×10⁶ cells/mL, about 2.2 μg per million cells of one or more cationic polymers, about 25% of stabilizer relative to the one or more cationic polymers, and about 1 μg per million cells of one or more nucleic acids.
